(19) United States
(12) Patent Application Publication
(10) Pub. No.: US 2010/0115660 A1
(43) Pub. Date: May 6, 2010

(54) Compositions and Methods Using RNA Interference of OPR3-Like Gene For Control of Nematodes

(75) Inventor: Aaron Wiig, Chapel Hill, NC (US)

Correspondence Address: BASF CORPORATION, CARL-BOSCH-STRASSE 38, LUDWIGSHAFEN, D67056, DE

(73) Assignee: BASF Plant Science GmbH, Ludwigshafen (DE)

(21) Appl. No.: 12/524,417

(22) PCT Filed: Feb. 5, 2008
(86) PCT No.: PCT/EP2008/051370

§ 371 Date: Jul. 24, 2009

Related U.S. Application Data
(60) Provisional application No. 60/900,146, filed on Feb. 8, 2007.

(51) Int. Cl. A01H 1/00; C07H 21/02; A01H 11/00; A01H 5/00

(52) U.S. Cl. 800/278; 536/23.1; 800/295; 800/298; 800/320.1; 800/320.3; 800/320; 800/320.2; 800/316; 800/312; 800/314; 800/313; 800/306; 800/318; 800/322

(57) ABSTRACT

The present invention concerns double stranded RNA compositions and transgenic plants capable of inhibiting expression of genes essential to establishing or maintaining nematode infestation in a plant, and methods associated therewith. Specifically, the invention relates to the use of RNA interference to inhibit expression of a target OPR3-like plant gene, and relates to the generation of plants that have increased resistance to parasitic nematodes.

### CROSS REFERENCE TO RELATED APPLICATIONS

This application claims the priority benefit of U.S. Provisional Application Ser. No. 60/900,146 filed Feb. 08, 2007.

### FIELD OF THE INVENTION

The invention relates to the control of nematodes. Disclosed herein are methods of producing transgenic plants with increased nematode resistance, expression vectors comprising polynucleotides conferring nematode resistance, and transgenic plants and seeds generated thereof.

### BACKGROUND OF THE INVENTION

Nematodes are microscopic roundworms that feed on the roots, leaves and stems of more than 2,000 row crops, vegetables, fruits, and ornamental plants, causing an estimated $100 billion crop loss worldwide. A variety of parasitic nematode species infect crop plants, including root-knot nematodes (RKN), cyst- and lesion-forming nematodes. Root-knot nematodes, which are characterized by causing root gall formation at feeding sites, have a relatively broad host range and are therefore pathogenic on a large number of crop species. The cyst- and lesion-forming nematode species have a more limited host range, but still cause considerable losses in susceptible crops.

Pathogenic nematodes are present throughout the United States, with the greatest concentrations occurring in the warm, humid regions of the South and West and in sandy soils. Soybean cyst nematode (Heterodera glycines), the most serious pest of soybean plants, was first discovered in the United States in North Carolina in 1954. Some areas are so heavily infested by soybean cyst nematode (SCN) that soybean production is no longer economically possible without control measures. Although soybean is the major economic crop attacked by SCN, SCN parasitizes some fifty hosts in total, including field crops, vegetables, ornamentals, and weeds.

Signs of nematode damage include stunting and yellowing of leaves, and wilting of the plants during hot periods. However, nematode infestation can cause significant yield losses without any obvious above-ground disease symptoms. The primary causes of yield reduction are due to root damage underground. Roots infected by SCN are dwarfed or stunted. Nematode infestation also can decrease the number of nitrogen-fixing nodules on the roots, and may make the roots more susceptible to attacks by other soil-borne plant pathogens.

The nematode life cycle has three major stages: egg, juvenile, and adult. The life cycle varies between species of nematodes. For example, the SCN life cycle can usually be completed in 24 to 30 days under optimum conditions whereas other species can take as long as a year, or longer, to complete the life cycle. When temperature and moisture levels become favorable in the spring, worm-shaped juveniles hatch from eggs in the soil. Only nematodes in the juvenile developmental stage are capable of infecting soybean roots.

The life cycle of SCN has been the subject of many studies, and as such are a useful example for understanding the nematode life cycle. After penetrating soybean roots, SCN juveniles move through the root until they contact vascular tissue, at which time they stop migrating and begin to feed. With a stylet, the nematode injects secretions that modify certain root cells and transform them into specialized feeding sites. The root cells are morphologically transformed into large multinucleate syncytia (or giant cells in the case of RKN), which are used as a source of nutrients for the nematodes. The actively feeding nematodes thus steal essential nutrients from the plant resulting in yield loss. As female nematodes feed, they swell and eventually become so large that their bodies break through the root tissue and are exposed on the surface of the root.

After a period of feeding, male SCN nematodes, which are not swollen as adults, migrate out of the root into the soil and fertilize the enlarged adult females. The males then die, while the females remain attached to the root system and continue to feed. The eggs in the swollen females begin developing, initially in a mass or egg sac outside the body, and then later within the nematode body cavity. Eventually the entire adult female body cavity is filled with eggs, and the nematode dies. It is the egg-filled body of the dead female that is referred to as the cyst. Cysts eventually dislodge and are found free in the soil. The walls of the cyst become very tough, providing excellent protection for the approximately 200 to 400 eggs contained within. SCN eggs survive within the cyst until proper hatching conditions occur. Although many of the eggs may hatch within the first year, many also will survive within the protective cysts for several years.

A nematode can move through the soil only a few inches per year on its own power. However, nematode infestation can be spread substantial distances in a variety of ways. Anything that can move infested soil is capable of spreading the infestation, including farm machinery, vehicles and tools, wind, water, animals, and farm workers. Seed sized particles of soil often contaminate harvested seed. Consequently, nematode infestation can be spread when contaminated seed from infested fields is planted in non-infested fields. There is even evidence that certain nematode species can be spread by birds. Only some of these causes can be prevented.

Traditional practices for managing nematode infestation include: maintaining proper soil nutrients and soil pH levels in nematode-infested land; controlling other plant diseases, as well as insect and weed pests; using sanitation practices such as plowing, planting, and cultivating of nematode-infested fields only after working non-infested fields; cleaning equipment thoroughly with high pressure water or steam after working in infested fields; not using seed grown on infested land for planting non-infested fields unless the seed has been properly cleaned; rotating infested fields and alternating host crops with non-host crops; using nematicides; and planting resistant plant varieties.

Methods have been proposed for the genetic transformation of plants in order to confer increased resistance to plant parasitic nematodes. U.S. Pat. Nos. 5,589,622 and 5,824,876 are directed to the identification of plant genes expressed specifically in or adjacent to the feeding site of the plant after attachment by the nematode. The promoters of these plant target genes can then be used to direct the specific expression of detrimental proteins or enzymes, or the expression of antisense RNA to the target gene or to general cellular genes. The plant promoters may also be used to confer nematode resistance specifically at the feeding site by transforming the plant with a construct comprising the promoter of the plant target gene linked to a gene whose product induces lethality in the nematode after ingestion.

Recently, RNA interference (RNAi), also referred to as gene silencing, has been proposed as a method for controlling nematodes. When double-stranded RNA (dsRNA) corresponding essentially to the sequence of a target gene or mRNA is introduced into a cell, expression from the target gene is inhibited (See e.g., U.S. Pat. No. 6,506,559). U.S. Pat. No. 6,506,559 demonstrates the effectiveness of RNAi against known genes in Caenorhabditis elegans, but does not demonstrate the usefulness of RNAi for controlling plant parasitic nematodes.

Use of RNAi to target essential nematode genes has been proposed, for example, in WO 01/96584, WO 01/37654, U.S. 2004/0098761, U.S. 2005/0091713, U.S. 2005/0188438, U.S. 2006/0037101, U.S. 2006/0080749, U.S. 2007/0199100, and U.S. 2007/0250947.

A number of models have been proposed for the action of RNAi. In mammalian systems, dsRNAs larger than 30 nucleotides trigger induction of interferon synthesis and a global shut-down of protein syntheses, in a non-sequence-specific manner. However, U.S. Pat. No. 6,506,559 discloses that in nematodes, the length of the dsRNA corresponding to the target gene sequence may be at least 25, 50, 100, 200, 300, or 400 bases, and that even larger dsRNAs were also effective at inducing RNAi in C. elegans. It is known that when hair-pin RNA constructs comprising double stranded regions ranging from 98 to 854 nucleotides were transformed into a number of plant species, the target plant genes were efficiently silenced. There is general agreement that in many organisms, including nematodes and plants, large pieces of dsRNA are cleaved into about 19-24 nucleotide fragments (siRNA) within cells, and that these siRNAs are the actual mediators of the RNAi phenomenon.

The OPR3 enzyme (12-oxyphytodienoate reductase) is involved in jasmonic acid (JA) biosynthesis. The OPR3 enzyme converts 12-oxo-cis-10,15-phytodienoate (OPDA) to 3-oxo-2-cis(cis-2-pentenyl)-cyclopentane-1-octanoate, which undergoes 3 rounds of beta oxidation to generate (+)-7-isojasmonate (JA). Arabidopsis opr3 mutant plants are unable to accumulate JA and are male sterile (Stintzi and Browse, 2000, PNAS. 97:10625-10630). Treating Arabidopsis opr3 plants with exogenous OPDA up-regulated several genes and disclosed two distinct signal pathways, one through CO|1 and one through an electrophile effect of the cyclopentones (Stintzi et al., 2001, PNAS 98:12317-12319). OPDA in concert with JA finetunes the expression of defense genes. Resistance to certain insects and fungi can occur in the absence of JA (Stintzi et al., 2001, PNAS 98:12837-42).

Although there have been numerous efforts to use RNAi to control plant parasitic nematodes, to date no transgenic nematode-resistant plant has been deregulated in any country. Accordingly, there continues to be a need to identify safe and effective compositions and methods for the controlling plant parasitic nematodes using RNAi, and for the production of plants having increased resistance to plant parasitic nematodes.

### SUMMARY OF THE INVENTION

The present inventors have discovered that the soybean target gene, 12-oxyphytodienate reductase-like (OPR3-like) also designated as 45174942 (SEQ ID NO: 1), is up-regulated in SCN-induced syncytia compared to uninfected root tissue. The present inventors have demonstrated that inhibition of OPR3 levels using RNAi affects the ability of the plant to resist nematode infestation.

In a first embodiment, therefore, the invention provides a double stranded RNA (dsRNA) molecule comprising a) a first strand comprising a sequence substantially identical to a portion of an OPR3-like gene and b) a second strand comprising a sequence substantially complementary to the first strand.

The invention is further embodied in a pool of dsRNA molecules comprising a multiplicity of RNA molecules each comprising a double stranded region having a length of about 19 to 24 nucleotides, wherein said RNA molecules are derived from a polynucleotide being substantially identical to a portion of an OPR3-like gene.

In another embodiment, the invention provides a transgenic nematode-resistant plant capable of expressing a dsRNA that is substantially identical to a portion of an OPR3-like gene.

In another embodiment, the invention provides a transgenic plant capable of expressing a pool of dsRNA molecules, wherein each dsRNA molecule comprises a double stranded region having a length of about 19-24 nucleotides and wherein the RNA molecules are derived from a polynucleotide substantially identical to a portion of an OPR3-like gene.

In another embodiment, the invention provides a method of making a transgenic plant capable of expressing a pool of dsRNA molecules each of which is substantially identical to a portion of an OPR3-like gene in a plant, said method comprising the steps of: a) preparing a nucleic acid having a region that is substantially identical to a portion of an OPR3-like gene, wherein the nucleic acid is able to form a double-stranded transcript of a portion of an OPR3-like gene once expressed in the plant; b) transforming a recipient plant with said nucleic acid; c) producing one or more transgenic offspring of said recipient plant; and d) selecting the offspring for expression of said transcript.

The invention further provides a method of conferring nematode resistance to a plant, said method comprising the steps of: a) preparing a nucleic acid having a region that is substantially identical to a portion of an OPR3-like gene, wherein the nucleic acid is able to form a double-stranded transcript of a portion of a OPR3-like gene once expressed in the plant; b) transforming a recipient plant with said nucleic acid; c) producing one or more transgenic off-spring of said recipient plant; and d) selecting the offspring for nematode resistance.

The invention further provides a expression cassette and an expression vector comprising a sequence substantially identical to a portion of an OPR3-like gene.

In another embodiment, the invention provides a method for controlling the infection of a plant by a parasitic nematode, comprising the steps of transforming the plant with a dsRNA molecule operably linked to a root-preferred, nematode inducible or feeding site-preferred pro-moter, whereby the dsRNA comprising one strand that is substantially identical to a portion of a target nucleic acid essential to the formation, development or support of the feeding site, in particular the formation, development or support of a syncytia or giant cell, thereby controlling the infection of the plant by the nematode by removing or functionally incapacitating the feeding site, syncytia or giant cell, wherein the target nucleic acid is an OPR3-like gene.

### BRIEF DESCRIPTION OF THE DRAWINGS

FIG. 1 shows the table of SEQ ID NOs assigned to corresponding sequences.

FIGS. 2a-2c show amino acid alignment of OPR3-like proteins: the full length GmOPR3-like protein (SEQ ID NO:30), Q9FEW9 from tomato (Seq ID NO: 8), the protein encoded by At2g06050 from Arabidopsis (SEQ ID NO: 10), AAY27752 from Hevea (SEQ ID NO: 12), EAZ42984 from rice (SEQ ID NO: 14), AAY26527 from maize (SEQ ID NO: 16), AAY26528 from maize (SEQ ID NO: 18), EG030595 from Arachis (SEQ ID NO: 20), the protein encoded by TA29350_{—}4113 from Solanum (SEQ ID NO: 22), the protein encoded by TA4283_{—}3760 from Prunus (SEQ ID NO: 24), the protein encoded by TA23750_{—}3635 from Gossypium (SEQ ID NO: 26) and the protein encoded by TA7248_{—}49390 from Coffea (SEQ ID NO:28). The alignment was performed in Vector NTI software suite (gap opening penalty =10, gap extension penalty =0.05, gap separation penalty =8). The full length GmOPR3-like sequence was determined through 5′ RACE PCR as described in Example 4.

FIG. 3 shows the global amino acid percent identity of exemplary OPR3-like genes: the full-length GmOPR3-like protein (SEQ ID NO:30), Q9FEW9 from tomato (Seq ID NO: 8), the protein encoded by At2g06050 from Arabidopsis (SEQ ID NO: 10), AAY27752 from Hevea (SEQ ID NO: 12), EAZ42984 from rice (SEQ ID NO: 14), AAY26527 from maize (SEQ ID NO: 16), AAY26528 from maize (SEQ ID NO: 18), EG030595 from Arachis (SEQ ID NO: 20), the protein encoded by TA29350_{—}4113 from Solanum (SEQ ID NO: 22), the protein encoded by TA4283_{—}3760 from Prunus (SEQ ID NO: 24), the protein encoded by TA23750_{—}3635 from Gossypium (SEQ ID NO: 26) and the protein encoded by TA7248_{—}49390 from Coffea (SEQ ID

NO:28). Pairwise alignments and percent identities were calculated using Needle of EMBOSS-4.0.0 (Needleman, S. B. and Wunsch, C. D. (1970) J. Mol. Biol. 48, 443-453).

FIG. 4 shows the global nucleotide percent identity of exemplary OPR3-like genes: the full-length GmOPR3-like DNA (SEQ ID NO:29), Q9FEW9 DNA from tomato (Seq ID NO: 7), At2g06050 DNA from Arabidopsis (SEQ ID NO: 9), AAY27752 DNA from Hevea (SEQ ID NO:

11), EAZ42984 DNA from rice (SEQ ID NO: 13), AAY26527 DNA from maize (SEQ ID NO: 15), AAY26528 DNA from maize (SEQ ID NO: 17), EG030595 DNA from Arachis (SEQ ID NO: 19), TA29350_{—}4113 DNA from Solanum (SEQ ID NO: 21), TA4283_{—}3760 DNA from Prunus (SEQ ID NO: 23), TA23750_{—}3635 DNA from Gossypium (SEQ ID NO: 25) and TA7248_{—}49390 DNA from Coffea (SEQ ID NO:27). Pairwise alignments and percent identities were calculated using Needle of EMBOSS-4.0.0.

FIGS. 5a-5j show various 21mers by nucleotide position possible for exemplary OPR3-like encoding polynucleotide of SEQ ID NO:1, 7, 9, 11, 13, 15, 17, 19, 21, 23, 25, 27, 29, or a polynucleotide sequence encoding an OPR3-like homolog.

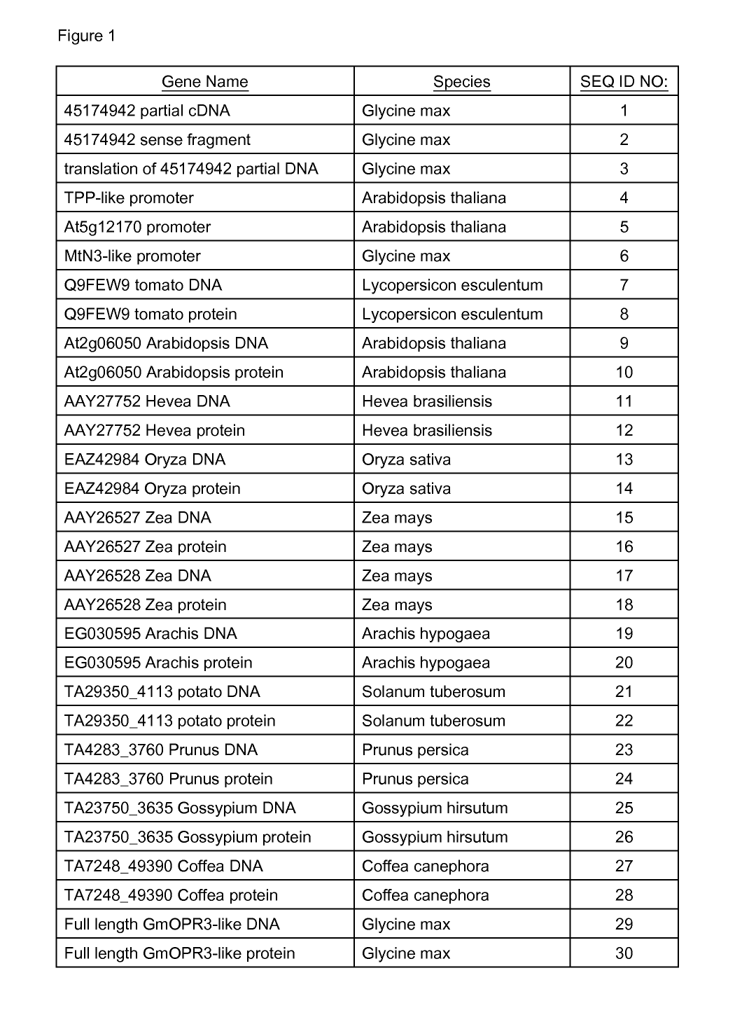

### DETAILED DESCRIPTION OF THE PREFERRED EMBODIMENTS

The present invention may be understood more readily by reference to the following detailed description of the preferred embodiments of the invention and the examples included herein. Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5^{th} Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized. It is to be understood that the terminology used herein is for the purpose of describing specific embodiments only and is not intended to be limiting.

Throughout this application, various patent and literature publications are referenced. The disclosures of all of these publications and those references cited within those publications in their entireties are hereby incorporated by reference into this application in order to more fully describe the state of the art to which this invention pertains.

A plant “OPR3-like gene” is defined herein as a gene having at least 60% sequence identity to a the 45174942 polynucleotide having the sequence as set forth in SEQ ID NO:1, which is the G. max OPR3-like gene. In accordance with the invention, OPR3-like genes include genes having sequences such as those set forth in SEQ ID NOs:2, 7, 9, 11, 13, 15, 17, 19, 21, 23, 25, 27, and 29, which are homologs of the G. max OPR3-like gene of SEQ ID NO:1. The OPR3-like genes defined herein encode polypeptides having at least 60% sequence identity to the G. max OPR3-like polypeptide having the sequence as set forth in SEQ ID NO:30. Such polypeptides include OPR3-like polypeptides having sequences as set forth in SEQ ID NOs: 8, 10, 12, 14, 16, 18, 20, 22, 24, 26, and 28

Additional OPR3-like genes (OPR3-like gene homologs) may be isolated from plants other than soybean using the information provided herein and techniques known to those of skill in the art of biotechnology. For example, a nucleic acid molecule from a plant that hybridizes under stringent conditions to the nucleic acid of SEQ ID NO:1 can be isolated from plant tissue cDNA libraries. Alternatively, mRNA can be isolated from plant cells (e.g., by the guanidinium-thiocyanate extraction procedure of Chirgwin et al., 1979, Biochemistry 18:5294-5299), and cDNA can be prepared using reverse transcriptase (e.g., Moloney MLV reverse transcriptase, available from Gibco/BRL, Bethesda, Md.; or AMV reverse transcriptase, available from Seika-gaku America, Inc., St. Petersburg, Fla.). Synthetic oligonucleotide primers for polymerase chain reaction amplification can be designed based upon the nucleotide sequence shown in SEQ ID NO:1. Additional oligonucleotide primers may be designed that are based on the sequences of the OPR3-like genes having the sequences as set forth in SEQ ID NOs: 2, 7, 9, 11, 13, 15, 17, 19, 21, 23, 25, 27, and 29. Nucleic acid molecules corresponding to the OPR3-like target genes defined herein can be amplified using cDNA or, alternatively, genomic DNA, as a template and appropriate oligonucleotide primers according to standard PCR amplification techniques. The nucleic acid molecules so amplified can be cloned into appropriate vectors and characterized by DNA sequence analysis.

As used herein, “RNAi” or “RNA interference” refers to the process of sequence-specific post-transcriptional gene silencing in plants, mediated by double-stranded RNA (dsRNA). As used herein, “dsRNA” refers to RNA that is partially or completely double stranded. Double stranded RNA is also referred to as small or short interfering RNA (sRNA), short interfering nucleic acid (siNA), short interfering RNA, micro-RNA (miRNA), and the like. In the RNAi process, dsRNA comprising a first strand that is substantially identical to a portion of a target gene, e.g. an OPR3-like gene, and a second strand that is complementary to the first strand is introduced into a plant. After introduction into the plant, the target gene-specific dsRNA is processed into relatively small fragments (siRNAs) and can subsequently become distributed throughout the plant, leading to a loss-of-function mutation having a phenotype that, over the period of a generation, may come to closely resemble the phenotype arising from a complete or partial deletion of the target gene. Alternatively, the target gene-specific dsRNA is operably associated with a regulatory element or promoter that results in expression of the dsRNA in a tissue, temporal, spatial or inducible manner and may further be processed into relatively small fragments by a plant cell containing the RNAi processing machinery, and the loss-of-function phenotype is obtained. Also, the regulatory element or promoter may direct expression preferentially to the roots or syncytia or giant cell where the dsRNA may be expressed either constitutively in those tissues or upon induction by the feeding of the nematode or juvenile nematode, such as J2 nematodes.

As used herein, taking into consideration the substitution of uracil for thymine when comparing RNA and DNA sequences, the term “substantially identical” as applied to dsRNA means that the nucleotide sequence of one strand of the dsRNA is at least about 80%-90% identical to 20 or more contiguous nucleotides of the target gene, more preferably, at least about 90-95% identical to 20 or more contiguous nucleotides of the target gene, and most preferably at least about 95%, 96%, 97%, 98% or 99% identical or absolutely identical to 20 or more contiguous nucleotides of the target gene. 20 or more nucleotides means a portion, being at least about 20, 21, 22, 23, 24, 25, 50, 100, 200, 300, 400, 500, 1000, 1500, or 2000 consecutive bases or up to the full length of the target gene.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. As used herein, the term “substantially complementary” means that two nucleic acid sequences are complementary over at least 80% of their nucleotides. Preferably, the two nucleic acid sequences are complementary over at least 85%, 90%, 95%, 96%, 97%, 98%, 99% or more or all of their nucleotides. Alternatively, “substantially complementary” means that two nucleic acid sequences can hybridize under high stringency conditions. As used herein, the term “substantially identical” or “corresponding to” means that two nucleic acid sequences have at least 80% sequence identity. Preferably, the two nucleic acid sequences have at least 85%, 90%, 95%, 96%, 97%, 98%, 99% or 100% of sequence identity.

Also as used herein, the terms “nucleic acid” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “control,” when used in the context of an infection, refers to the reduction or prevention of an infection. Reducing or preventing an infection by a nematode will cause a plant to have increased resistance to the nematode; however, such increased resistance does not imply that the plant necessarily has 100% resistance to infection. In preferred embodiments, the resistance to infection by a nematode in a resistant plant is greater than 10%, 20%, 30%, 40%, 50%, 60%, 70%, 80%, 90%, or 95% in comparison to a wild type plant that is not resistant to nematodes. Preferably the wild type plant is a plant of a similar, more preferably identical genotype as the plant having increased resistance to the nematode, but does not comprise a dsRNA directed to the target gene. The plant's resistance to infection by the nematode may be due to the death, sterility, arrest in development, or impaired mobility of the nematode upon exposure to the plant comprising dsRNA specific to a gene essential for development or maintenance of a functional feeding site, syncytia, or giant cell. The term “resistant to nematode infection” or “a plant having nematode resistance” as used herein refers to the ability of a plant, as compared to a wild type plant, to avoid infection by nematodes, to kill nematodes or to hamper, reduce or stop the development, growth or multiplication of nematodes. This might be achieved by an active process, e.g. by producing a substance detrimental to the nematode, or by a passive process, like having a reduced nutritional value for the nematode or not developing structures induced by the nematode feeding site like syncytia or giant cells. The level of nematode resistance of a plant can be determined in various ways, e.g. by counting the nematodes being able to establish parasitism on that plant, or measuring development times of nematodes, proportion of male and female nematodes or, for cyst nematodes, counting the number of cysts or nematode eggs produced on roots of an infected plant or plant assay system.

The term “plant” is intended to encompass plants at any stage of maturity or development, as well as any tissues or organs (plant parts) taken or derived from any such plant unless otherwise clearly indicated by context. Plant parts include, but are not limited to, stems, roots, flowers, ovules, stamens, seeds, leaves, embryos, meristematic regions, callus tissue, anther cultures, gametophytes, sporophytes, pollen, microspores, protoplasts, hairy root cultures, and the like. The present invention also includes seeds produced by the plants of the present invention. In one embodiment, the seeds are true breeding for an increased resistance to nematode infection as compared to a wild-type variety of the plant seed. As used herein, a “plant cell” includes, but is not limited to, a protoplast, gamete producing cell, and a cell that regenerates into a whole plant. Tissue culture of various tissues of plants and regeneration of plants therefrom is well known in the art and is widely published.

As used herein, the term “transgenic” refers to any plant, plant cell, callus, plant tissue, or plant part that contains all or part of at least one recombinant polynucleotide. In many cases, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

As used herein, the term “amount sufficient to inhibit expression” refers to a concentration or amount of the dsRNA that is sufficient to reduce levels or stability of mRNA or protein produced from a target gene in a plant. As used herein, “inhibiting expression” refers to the absence or observable decrease in the level of protein and/or mRNA product from a target gene. Inhibition of target gene expression may be lethal to the parasitic nematode either directly or indirectly through modification or eradication of the feeding site, syncytia, or giant cell, or such inhibition may delay or prevent entry into a particular developmental step (e.g., metamorphosis), if access to a fully functional feeding site, syncytia, or giant cell is associated with a particular stage of the parasitic nematode's life cycle. The consequences of inhibition can be confirmed by examination of the plant root for reduction or elimination of cysts or other properties of the nematode or nematode infestation (as presented below in Example 3).

In accordance with the invention, a plant is transformed with a nucleic acid or a dsRNA, which specifically inhibits expression of OPR3-like gene in the plant that is essential for the development or maintenance of a feeding site, syncytia, or giant cell; ultimately affecting the survival, metamorphosis, or reproduction of the nematode. In one embodiment, the dsRNA is encoded by a vector that has been transformed into an ancestor of the infected plant. Preferably, the nucleic acid sequence expressing said dsRNA is under the transcriptional control of a root specific promoter or a parasitic nematode feeding cell-specific promoter or a nematode inducible promoter.

Accordingly, the dsRNA of the invention comprises a first strand that is substantially identical to a portion of the OPR3-like target gene of a plant genome, and a second strand that is substantially complementary to the first strand. In preferred embodiments, the target gene is selected from the group consisting of: a) a polynucleotide comprising a sequence as set forth in SEQ ID NO:1, 2, 7, 9, 11, 13, 15, 17, 19, 21, 23, 25, 27, or 29; b) a polynucleotide encoding a polypeptide having a sequence as set forth in SEQ ID NO:3, 8, 10, 12, 14, 16, 18, 20, 22, 24, 26, 28, or 30; and c) a polynucleotide having 70% sequence identity to a polynucleotide having a sequence as set forth in SEQ ID NO:1, 2, 7, 9, 11, 13, 15, 17, 19, 21, 23, 25, 27, or 29. The length of the substantially identical double-stranded nucleotide sequences may be at least about 19, 20, 21, 22, 23, 24, 25, 50, 100, 200, 300, 400, 500, 1000, 1500, consecutive bases or up to the whole length of the OPR3-like gene. In a preferred embodiment, the length of the double-stranded nucleotide sequence is from approximately from about 19 to about 200-500 consecutive nucleotides in length. In another preferred embodiment, the dsRNA of the invention is substantially identical or is identical to bases 1 to 229 of SEQ ID NO: 2.

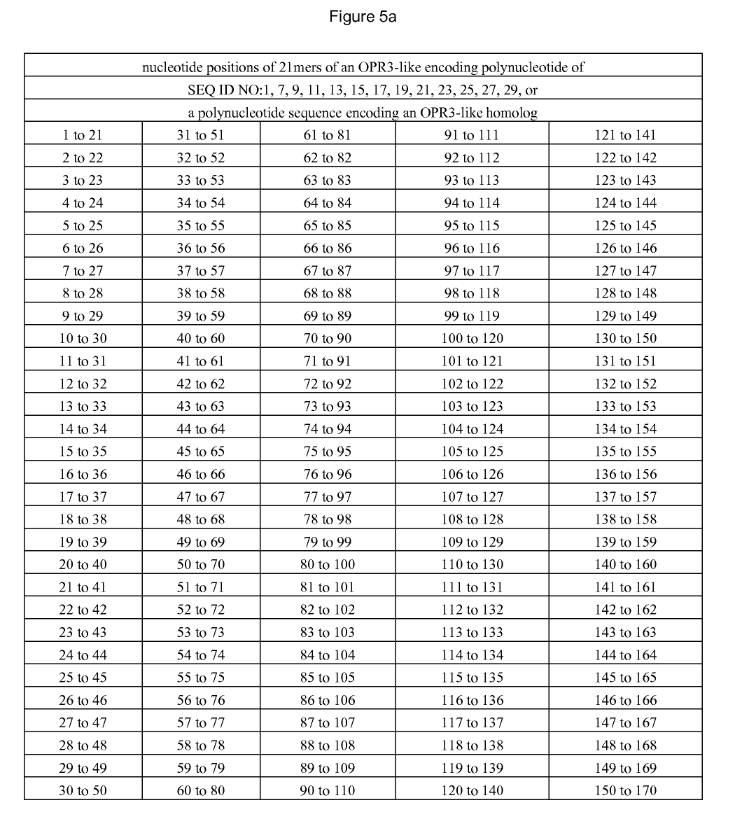

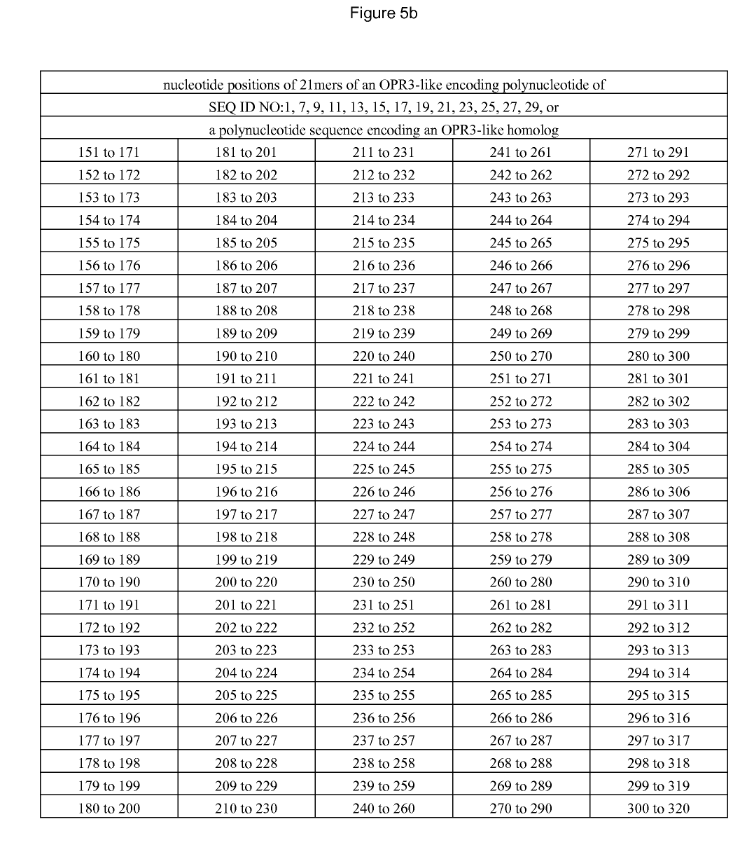

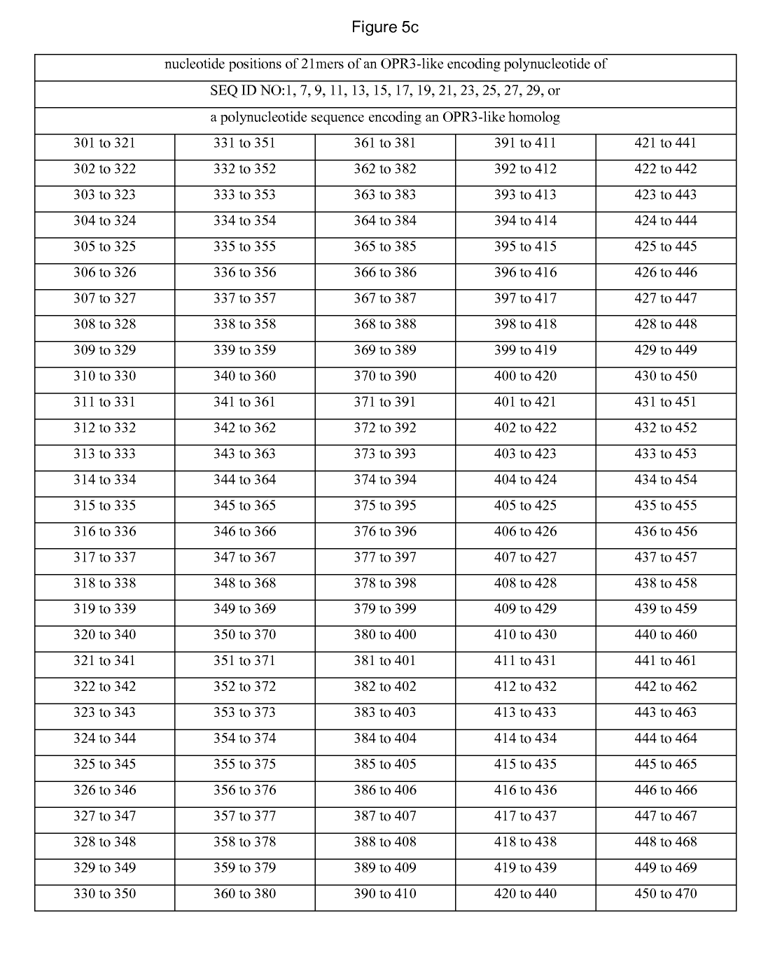

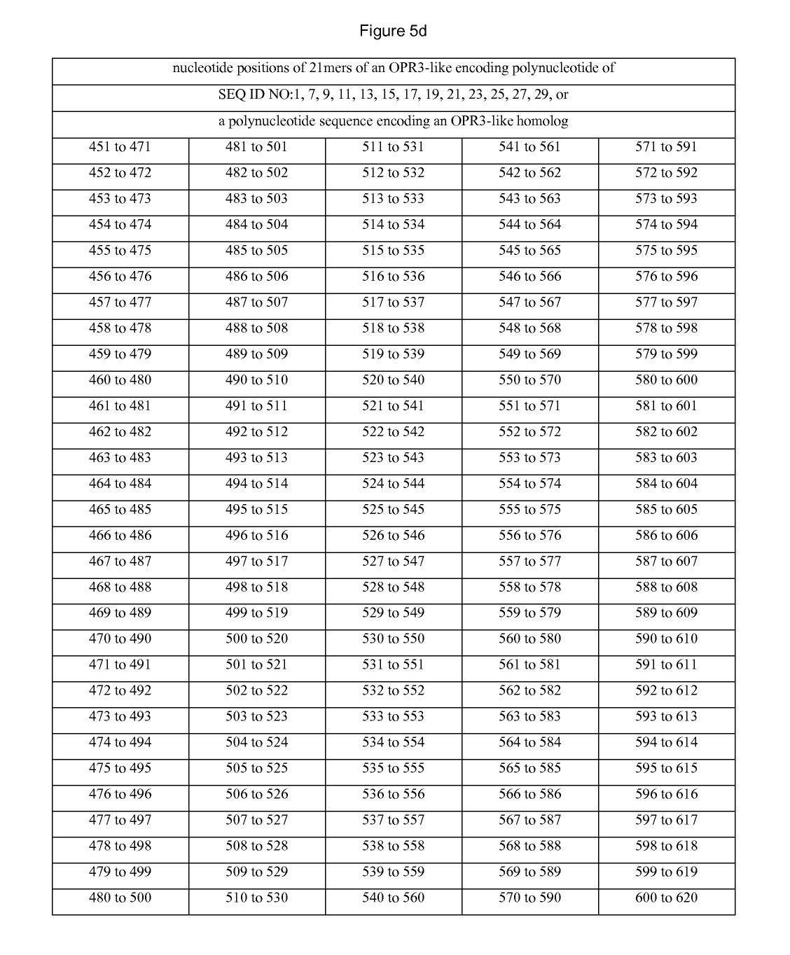

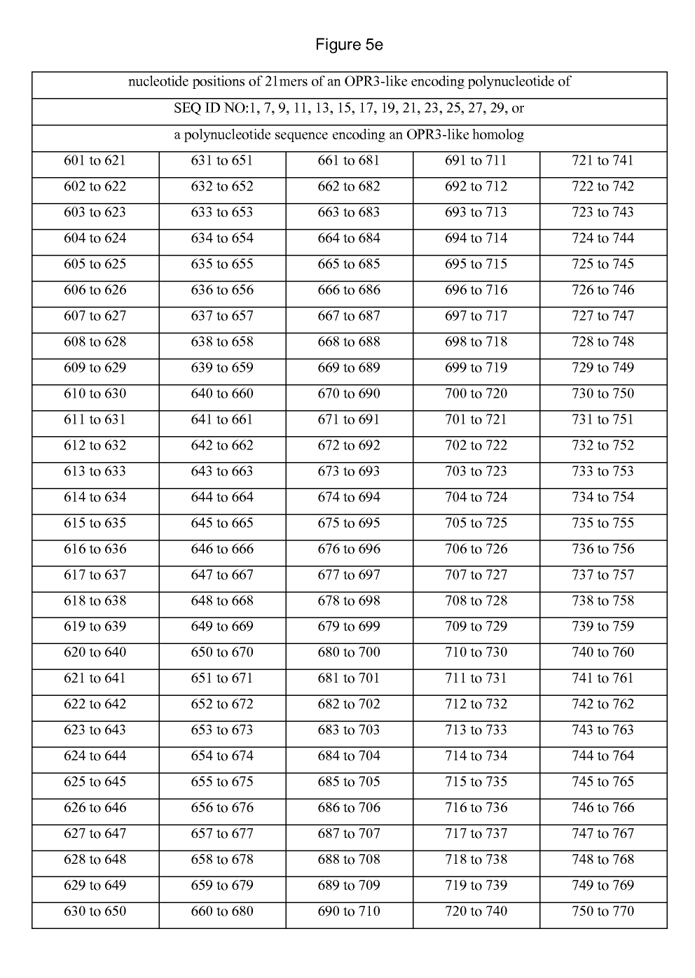

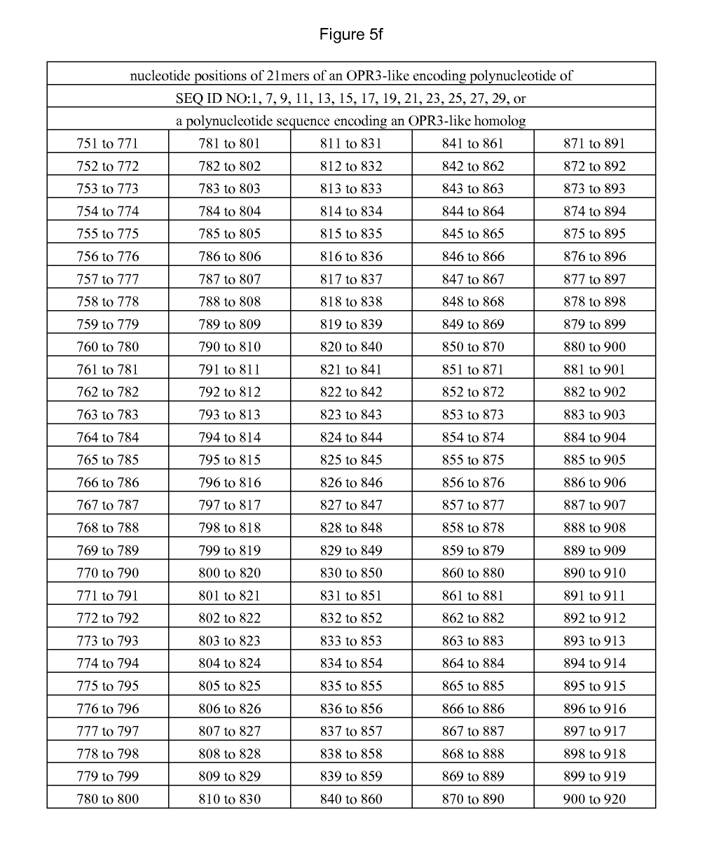

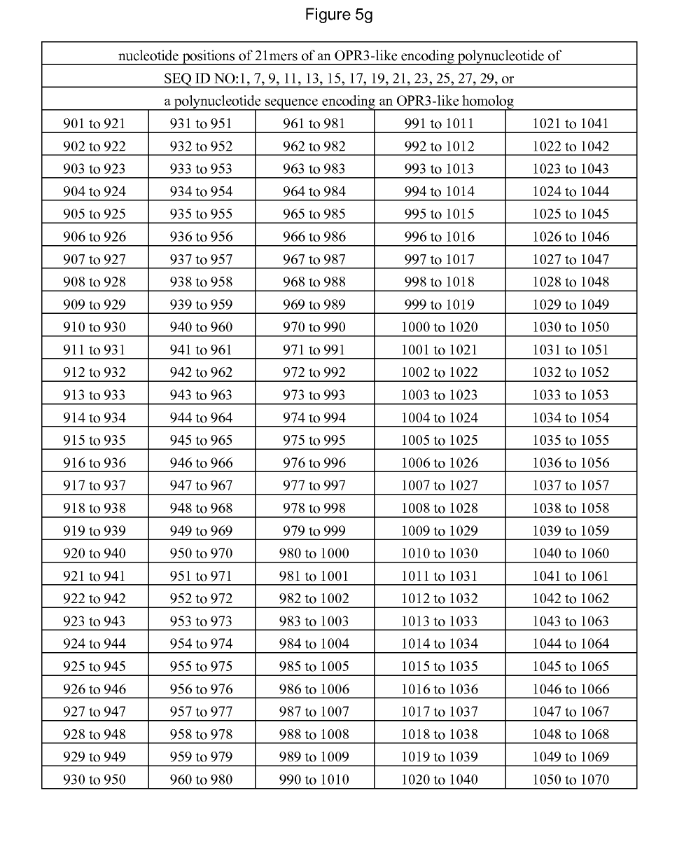

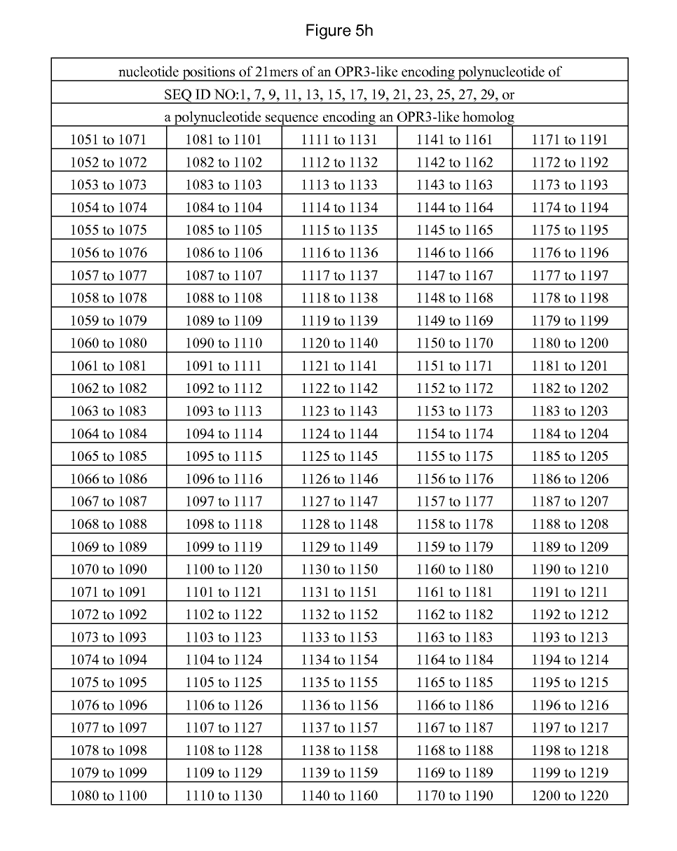

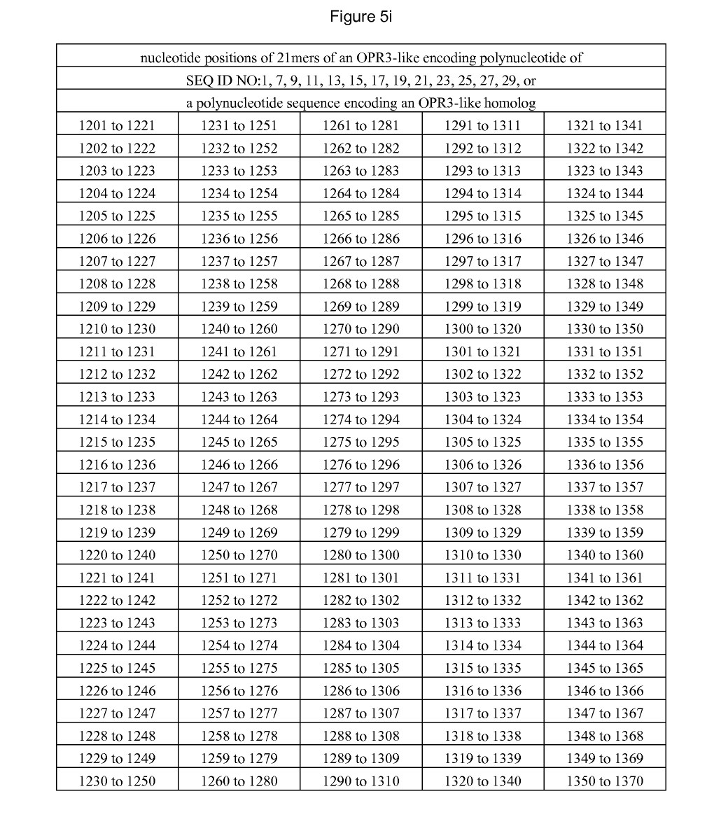

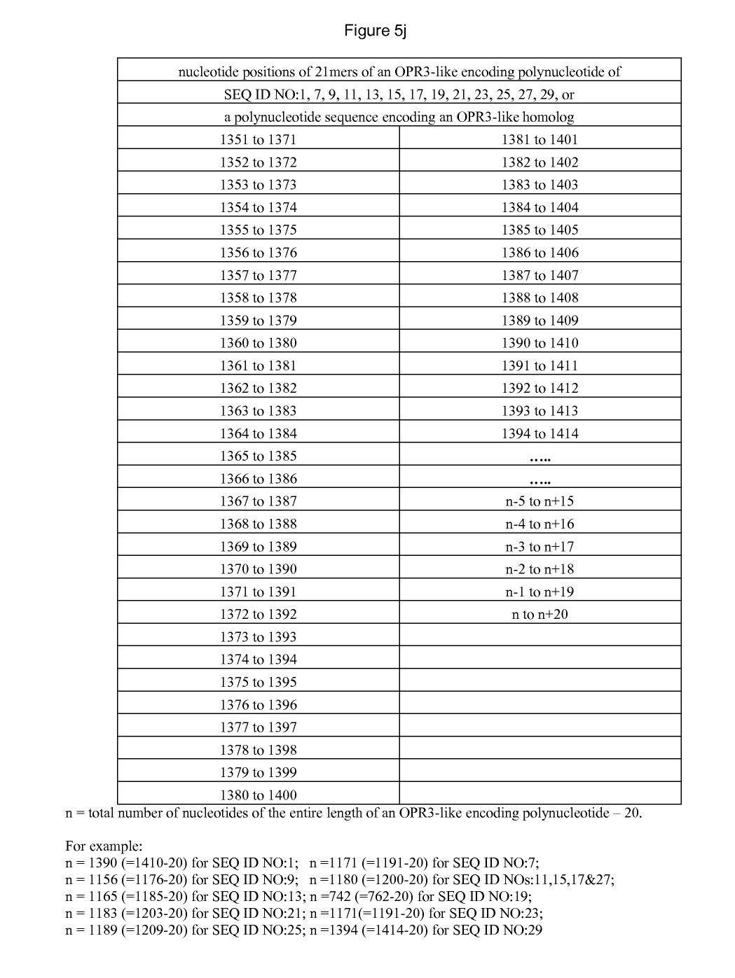

As discussed above, fragments of dsRNA larger than about 19-24 nucleotides in length are cleaved intracellularly by nematodes and plants to siRNAs of about 19-24 nucleotides in length, and these siRNAs are the actual mediators of the RNAi phenomenon. The table in FIGS. 5a-5j sets forth exemplary 21-mers of the OPR3-like genes defined herein. This table can also be used to calculate the 19, 20, 22, 23 or 24-mers by adding or subtracting the appropriate number of nucleotides from each 21 mer. Thus the dsRNA of the present invention may range in length from about 21 nucleotides to 200 nucleotides. Preferably, the dsRNA of the invention has a length from about 21 nucleotides to 600 consecutive nucleotides or up to the whole length of the OPR3-like gene. More preferably, the dsRNA of the invention has a length from about 21 nucleotides to 500 consecutive nucleotides, or from about 21 nucleotides to about 200 consecutive nucleotides.

As disclosed herein, 100% sequence identity between the RNA and the target gene is not required to practice the present invention. While a dsRNA comprising a nucleotide sequence identical to a portion of the OPR3-like gene is preferred for inhibition, the invention can tolerate sequence variations that might be expected due to gene manipulation or synthesis, genetic mutation, strain polymorphism, or evolutionary divergence. Thus the dsRNAs of the invention also encompass dsRNAs comprising a mismatch with the target gene of at least 1, 2, or more nucleotides. For example, it is contemplated in the present invention that the 21 mer dsRNA sequences exemplified in FIGS. 5a-5j may contain an addition, deletion or substitution of 1, 2, or more nucleotides, so long as the resulting sequence still interferes with the OPR3-like gene function.

Sequence identity between the dsRNAs of the invention and the OPR3-like target genes may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 80% sequence identity, 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript under stringent conditions (e.g., 400 mM NaCl, 40 mM PIPES pH 6.4, 1 mM EDTA, 60° C. hybridization for 12-16 hours; followed by washing at 65° C. with 0.1% SDS and 0.1% SSC for about 15-60 minutes).

When dsRNA of the invention has a length longer than about 21 nucleotides, for example from 50 nucleotides to 1000 nucleotides, it will be cleaved randomly to dsRNAs of about 21 nucleotides within the plant or parasitic nematode cell, the siRNAs. The cleavage of a longer dsRNA of the invention will yield a pool of about 21mer dsRNAs (ranging from 19mers to 24mers), derived from the longer dsRNA. This pool of about 21mer dsRNAs is also encompassed within the scope of the present invention, whether generated intracellularly within the plant or nematode or synthetically using known methods of oligonucleotide synthesis.

The siRNAs of the invention have sequences corresponding to fragments of 19-24 contiguous nucleotides across the entire sequence of an OPR3-like target gene. For example, a pool of siRNA of the invention derived from the OPR3-like genes as set forth in SEQ ID NO: 1, 2, 7, 9, 11, 13, 15, 17, 19, 21, 23, 25, 27, or 29may comprise a multiplicity of RNA molecules which are selected from the group consisting of oligonucleotides comprising one strand which is substantially identical to the 21 mer nucleotides of SEQ ID NO: 1, 2, 7, 9, 11, 13, 15, 17, 19, 21, 23, 25, 27, or 29 found in FIGS. 5a-5j. A pool of siRNA of the invention derived from OPR3-like genes described by SEQ ID NO: 1, 2, 7, 9, 11, 13, 15, 17, 19, 21, 23, 25, 27, or 29 may also comprise any combination of the specific RNA molecules having any of the 21 contiguous nucleotide sequences derived from SEQ ID NO: 1, 2, 7, 9, 11, 13, 15, 17, 19, 21, 23, 25, 27, or 29 set forth in FIGS. 5a-5j. Further, as noted above, multiple specialized Dicers in plants generate siRNAs typically ranging in size from 19 nt to 24 nt (See Henderson et al., 2006. Nature Genetics 38:721-725.). The siRNAs of the present invention may range from about 19 contiguous nucleotide sequences to about 24 contiguous nucleotide sequences. Similarly, a pool of siRNA of the invention may comprise a multiplicity of RNA molecules having any of about 19, 20, 21, 22, 23, or 24 contiguous nucleotide sequences derived from SEQ ID NO: 1, 2, 5, 6, 8, 10, 12, 14, 16, 18, 20, 22, 24 or 26. Alternatively, the pool of siRNA of the invention may comprise a multiplicity of RNA molecules having a combination of any of about 19, 20, 21, 22, 23, and/or 24 contiguous nucleotide sequences derived from SEQ ID NO: 1, 2, 7, 9, 11, 13, 15, 17, 19, 21, 23, 25, 27, or 29.

The dsRNA of the invention may optionally comprise a single stranded overhang at either or both ends. The double-stranded structure may be formed by a single self-complementary RNA strand (i.e. forming a hairpin loop) or two complementary RNA strands. RNA duplex formation may be initiated either inside or outside the cell. When the dsRNA of the invention forms a hairpin loop, it may optionally comprise an intron, as set forth in US 2003/0180945A1 or a nucleotide spacer, which is a stretch of sequence between the complementary RNA strands to stabilize the hairpin transgene in cells. Methods for making various dsRNA molecules are set forth, for example, in WO 99/53050 and in U.S. Pat. No. 6,506,559. The RNA may be introduced in an amount that allows delivery of at least one copy per cell. Higher doses of double-stranded material may yield more effective inhibition.

In another embodiment, the invention provides an isolated recombinant expression vector comprising a nucleic acid encoding a dsRNA molecule as described above, wherein expression of the vector in a host plant cell results in increased resistance to a parasitic nematode as compared to a wild-type variety of the host plant cell. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host plant cell into which they are introduced. Other vectors are integrated into the genome of a host plant cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., potato virus X, tobacco rattle virus, and Geminivirus), which serve equivalent functions.

The recombinant expression vectors of the invention comprise a nucleic acid of the invention in a form suitable for expression of the nucleic acid in a host plant cell, which means that the recombinant expression vector includes one or more regulatory sequences, e.g. promoters, selected on the basis of the host plant cells to be used for expression, which is operatively linked to the nucleic acid sequence to be expressed. With respect to a recombinant expression vector, the terms “operatively linked” and “in operative association” are interchangeable and are intended to mean that the nucleotide sequence of interest is linked to the regulatory sequence(s) in a manner which allows for expression of the nucleotide sequence (e.g., in a host plant cell when the vector is introduced into the host plant cell). The term “regulatory sequence” is intended to include promoters, enhancers, and other expression control elements (e.g., polyadenylation signals). Such regulatory sequences are described, for example, in Goeddel, Gene Expression Technology: Methods in Enzymology 185, Academic Press, San Diego, Calif. (1990) and Gruber and Crosby, in: Methods in Plant Molecular Biology and Biotechnology, Eds. Glick and Thompson, Chapter 7, 89-108, CRC Press: Boca Raton, Fla., including the references therein. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of dsRNA desired, etc. The expression vectors of the invention can be introduced into plant host cells to thereby produce dsRNA molecules of the invention encoded by nucleic acids as described herein.

In accordance with the invention, the recombinant expression vector comprises a regulatory sequence operatively linked to a nucleotide sequence that is a template for one or both strands of the dsRNA molecules of the invention. In one embodiment, the nucleic acid molecule further comprises a promoter flanking either end of the nucleic acid molecule, wherein the promoters drive expression of each individual DNA strand, thereby generating two complementary RNAs that hybridize and form the dsRNA. In another embodiment, the nucleic acid molecule comprises a nucleotide sequence that is transcribed into both strands of the dsRNA on one transcription unit, wherein the sense strand is transcribed from the 5′ end of the transcription unit and the antisense strand is transcribed from the 3′ end, wherein the two strands are separated by about 3 to about 500 base pairs or more, and wherein after transcription, the RNA transcript folds on itself to form a hairpin. In accordance with the invention, the spacer region in the hairpin transcript may be any DNA fragment.

According to the present invention, the introduced polynucleotide may be maintained in the plant cell stably if it is incorporated into a non-chromosomal autonomous replicon or integrated into the plant chromosomes. Alternatively, the introduced polynucleotide may be present on an extra-chromosomal non-replicating vector and be transiently expressed or transiently active. Whether present in an extra-chromosomal non-replicating vector or a vector that is integrated into a chromosome, the polynucleotide preferably resides in a plant expression cassette. A plant expression cassette preferably contains regulatory sequences capable of driving gene expression in plant cells that are operatively linked so that each sequence can fulfill its function, for example, termination of transcription by polyadenylation signals. Preferred polyadenylation signals are those originating from Agrobacterium tumefaciens t-DNA such as the gene 3 known as octopine synthase of the Ti-plasmid pTiACH5 (Gielen et al., 1984, EMBO J. 3:835) or functional equivalents thereof, but also all other terminators functionally active in plants are suitable. As plant gene expression is very often not limited at transcriptional levels, a plant expression cassette preferably contains other operatively linked sequences like translational enhancers such as the overdrive-sequence containing the 5′-untranslated leader sequence from tobacco mosaic virus enhancing the polypeptide per RNA ratio (Gallie et al., 1987, Nucl. Acids Research 15:8693-8711). Examples of plant expression vectors include those detailed in: Becker, D. et al., 1992, New plant binary vectors with selectable markers located proximal to the left border, Plant Mol. Biol. 20:1195-1197; Bevan, M. W., 1984, Binary Agrobacterium vectors for plant transformation, Nucl. Acid. Res. 12:8711-8721; and Vectors for Gene Transfer in Higher Plants; in: Transgenic Plants, Vol. 1, Engineering and Utilization, eds.: Kung and R. Wu, Academic Press, 1993, S. 15-38.

Plant gene expression should be operatively linked to an appropriate promoter conferring gene expression in a temporal-preferred, spatial-preferred, cell type-preferred, and/or tissue-preferred manner. Promoters useful in the expression cassettes of the invention include any promoter that is capable of initiating transcription in a plant cell present in the plant's roots. Such promoters include, but are not limited to those that can be obtained from plants, plant viruses and bacteria that contain genes that are expressed in plants, such as Agrobacterium and Rhizobium. Preferably, the expression cassette of the invention comprises a root-specific promoter, a pathogen inducible promoter, or a nematode inducible promoter. More preferably the nematode inducible promoter is a parasitic nematode feeding site-specific promoter. A parasitic nematode feeding site-specific promoter may be specific for syncytial cells or giant cells or specific for both kinds of cells. A promoter is inducible, if its activity, measured on the amount of RNA produced under control of the promoter, is at least 30%, 40%, 50% preferably at least 60%, 70%, 80%, 90% more preferred at least 100%, 200%, 300% higher in its induced state, than in its un-induced state. A promoter is cell-, tissue- or organ-specific, if its activity , measured on the amount of RNA produced under control of the promoter, is at least 30%, 40%, 50% preferably at least 60%, 70%, 80%, 90% more preferred at least 100%, 200%, 300% higher in a particular cell-type, tissue or organ, then in other cell-types or tissues of the same plant, preferably the other cell-types or tissues are cell types or tissues of the same plant organ, e.g. a root. In the case of organ specific promoters, the promoter activity has to be compared to the promoter activity in other plant organs, e.g. leaves, stems, flowers or seeds.

The promoter may be constitutive, inducible, developmental stage-preferred, cell type-preferred, tissue-preferred or organ-preferred. Constitutive promoters are active under most conditions. Non-limiting examples of constitutive promoters include the CaMV 19S and 35S promoters (Odell et al., 1985, Nature 313:810-812), the sX CaMV 35S promoter (Kay et al., 1987, Science 236:1299-1302), the Sep1 promoter, the rice actin promoter (McElroy et al., 1990, Plant Cell 2:163-171), the Arabidopsis actin promoter, the ubiquitin promoter (Christensen et al., 1989, Plant Molec. Biol. 18:675-689); pEmu (Last et al., 1991, Theor. Appl. Genet. 81:581-588), the figwort mosaic virus 35S promoter, the Smas promoter (Velten et al., 1984, EMBO J. 3:2723-2730), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like. Promoters that express the dsRNA in a cell that is contacted by parasitic nematodes are preferred. Alternatively, the promoter may drive expression of the dsRNA in a plant tissue remote from the site of contact with the nematode, and the dsRNA may then be transported by the plant to a cell that is contacted by the parasitic nematode in particular cells of, or close by nematode feeding sites, e.g. syncytial cells or giant cells.

Inducible promoters are active under certain environmental conditions, such as the presence or absence of a nutrient or metabolite, heat or cold, light, pathogen attack, anaerobic conditions, and the like. For example, the promoters TobRB7, AtRPE, AtPyk10, Geminil9, and AtHMG1 have been shown to be induced by nematodes (for a review of nematode-inducible promoters, see Ann. Rev. Phytopathol. (2002) 40:191-219; see also U.S. Pat. No. 6,593,513).

Method for isolating additional promoters, which are inducible by nematodes are set forth in U.S. Pat. Nos. 5,589,622 and 5,824,876. Other inducible promoters include the hsp80 promoter from Brassica, being inducible by heat shock; the PPDK promoter is induced by light; the PR-1 promoter from tobacco, Arabidopsis, and maize are inducible by infection with a pathogen; and the Adh1 promoter is induced by hypoxia and cold stress. Plant gene expression can also be facilitated via an inducible promoter (For review, see Gatz, 1997, Annu. Rev. Plant Physiol. Plant Mol. Biol. 48:89-108). Chemically inducible promoters are especially suitable if time-specific gene expression is desired. Non-limiting examples of such promoters are a salicylic acid inducible promoter (PCT Application No. WO 95/19443), a tetracycline inducible promoter (Gatz et al., 1992, Plant J. 2:397-404) and an ethanol inducible promoter (PCT Application No. WO 93/21334).

Developmental stage-preferred promoters are preferentially expressed at certain stages of development. Tissue and organ preferred promoters include those that are preferentially expressed in certain tissues or organs, such as leaves, roots, seeds, or xylem. Examples of tissue preferred and organ preferred promoters include, but are not limited to fruit-preferred, ovule-preferred, male tissue-preferred, seed-preferred, integument-preferred, tuber-preferred, stalk-preferred, pericarp-preferred, and leaf-preferred, stigma-preferred, pollen-preferred, anther-preferred, a petal-preferred, sepal-preferred, pedicel-preferred, silique-preferred, stem-preferred, root-preferred promoters and the like. Seed preferred promoters are preferentially expressed during seed development and/or germination. For example, seed preferred promoters can be embryo-preferred, endosperm preferred and seed coat-preferred. See Thompson et al., 1989, BioEssays 10:108. Examples of seed preferred promoters include, but are not limited to cellulose synthase (celA), Cim1, gamma-zein, globulin-1, maize 19 kD zein (cZ19B1) and the like.

Other suitable tissue-preferred or organ-preferred promoters include, but are not limited to, the napin-gene promoter from rapeseed (U.S. Pat. No. 5,608,152), the USP-promoter from Vicia faba (Baeumlein et al., 1991, Mol Gen Genet. 225(3):459-67), the oleosin-promoter from Arabidopsis (PCT Application No. WO 98/45461), the phaseolin-promoter from Phaseolus vulgaris (U.S. Pat. No. 5,504,200), the Bce4-promoter from Brassica (PCT Application No. WO 91/13980), or the legumin B4 promoter (LeB4; Baeumlein et al., 1992, Plant Journal, 2(2):233-9), as well as promoters conferring seed specific expression in monocot plants like maize, barley, wheat, rye, rice, etc. Suitable promoters to note are the Ipt2 or Ipt1-gene promoter from barley (PCT Application No. WO 95/15389 and PCT Application No. WO 95/23230) or those described in PCT Application No. WO 99/16890 (promoters from the barley hordein-gene, rice glutelin gene, rice oryzin gene, rice prolamin gene, wheat gliadin gene, wheat glutelin gene, oat glutelin gene, Sorghum kasirin-gene, and rye secalin gene).

Other promoters useful in the expression cassettes of the invention include, but are not limited to, the major chlorophyll a/b binding protein promoter, histone promoters, the Ap3 promoter, the β-conglycin promoter, the napin promoter, the soybean lectin promoter, the maize 15 kD zein promoter, the 22 kD zein promoter, the 27 kD zein promoter, the g-zein promoter, the waxy, shrunken 1, shrunken 2, and bronze promoters, the Zm13 promoter (U.S. Pat. No. 5,086,169), the maize polygalacturonase promoters (PG) (U.S. Pat. Nos. 5,412,085 and 5,545,546), and the SGB6 promoter (U.S. Pat. No. 5,470,359), as well as synthetic or other natural promoters.

In accordance with the present invention, the expression cassette comprises an expression control sequence operatively linked to a nucleotide sequence that is a template for one or both strands of the dsRNA. The dsRNA template comprises (a) a first stand having a sequence substantially identical to from about 19 to 500, or up to the full length, consecutive nucleotides of an OPR3-like gene; and (b) a second strand having a sequence substantially complementary to the first strand. In further embodiments, a promoter flanks either end of the template nucleotide sequence, wherein the promoters drive expression of each individual DNA strand, thereby generating two complementary RNAs that hybridize and form the dsRNA. In alternative embodiments, the nucleotide sequence is transcribed into both strands of the dsRNA on one transcription unit, wherein the sense strand is transcribed from the 5′ end of the transcription unit and the anti-sense strand is transcribed from the 3′ end, wherein the two strands are separated by 3 to 500 base pairs, and wherein after transcription, the RNA transcript folds on itself to form a hairpin.

In another embodiment, the vector contains a bidirectional promoter, driving expression of two nucleic acid molecules, whereby one nucleic acid molecule codes for the sequence substantially identical to a portion of a OPR3-like gene and the other nucleic acid molecule codes for a second sequence being substantially complementary to the first strand and capable of forming a dsRNA, when both sequences are transcribed. A bidirectional promoter is a promoter capable of mediating expression in two directions.

In another embodiment, the vector contains two promoters one mediating transcription of the sequence substantially identical to a portion of a OPR3-like gene and another promoter mediating transcription of a second sequence being substantially complementary to the first strand and capable of forming a dsRNA, when both sequences are transcribed. The second promoter might be a different promoter.

A different promoter means a promoter having a different activity in regard to cell or tissue specificity, or showing expression on different inducers for example, pathogens, abiotic stress or chemicals. For example, one promoter might by constitutive or tissue specific and another might be tissue specific or inducible by pathogens. In one embodiment one promoter mediates the transcription of one nucleic acid molecule suitable for overexpression of an OPR3-like gene, while another promoter mediates tissue- or cell-specific transcription or pathogen inducible expression of the complementary nucleic acid.

The invention is also embodied in a transgenic plant capable of expressing the dsRNA of the invention and thereby inhibiting the target genes e.g. in the roots, feeding site, syncytia and/or giant cell. The plant or transgenic plant may be any plant, such like, but not limited to trees, cut flowers, ornamentals, vegetables or crop plants. The plant may be from a genus selected from the group consisting of Medicago, Lycopersicon, Brassica, Cucumis, Solanum, Juglans, Gossypium, Malus, Vitis, Antirrhinum, Populus, Fragaria, Arabidopsis, Picea, Capsicum, Chenopodium, Dendranthema, Pharbitis, Pinus, Pisum, Oryza, Zea, Triticum, Triticale, Secale, Lolium, Hordeum, Glycine, Pseudotsuga, Kalanchoe, Beta, Helianthus, Nicotiana, Cucurbita, Rosa, Fragaria, Lotus, Medicago, Onobrychis, trifolium, Trigonella, Vigna, Citrus, Linum, Geranium, Manihot, Daucus, Raphanus, Sinapis, Atropa, Datura, Hyoscyamus, Nicotiana, Petunia, Digitalis, Majorana, Ciahorium, Lactuca, Bromus, Asparagus, Antirrhinum, Heterocallis, Nemesis, Pelargonium, Panieum, Pennisetum, Ranunculus, Senecio, Salpiglossis, Browaalia, Phaseolus, Avena, and Allium, or the plant may be selected from a genus selected from the group consisting of Arabidopsis, Medicago, Lycopersicon, Brassica, Cucumis, Solanum, Juglans, Gossypium, Malus, Vitis, Antirrhinum, Brachipodium, Populus, Fragaria, Arabidopsis, Picea, Capsicum, Chenopodium, Dendranthema, Pharbitis, Pinus, Pisum, Oryza, Zea, Triticum, Triticale, Secale, Lolium, Hordeum, Glycine, Pseudotsuga, Kalanchoe, Beta, Helianthus, Nicotiana, Cucurbita, Rosa, Fragaria, Lotus, Medicago, Onobrychis, trifolium, Trigonella, Vigna, Citrus, Linum, Geranium, Manihot, Daucus, Raphanus, Sinapis, Atropa, Datura, Hyoscyamus, Nicotiana, Petunia, Digitalis, Majorana, Ciahorium, Lactuca, Bromus, Asparagus, Antirrhinum, Heterocallis, Nemesis, Pelargonium, Panicum, Pennisetum, Ranunculus, Senecio, Salpiglossis, Browaalia, Phaseolus, Avena, and Allium. In one embodiment the plant is a monocotyledonous plant or a dicotyledonous plant.

In another embodiment the plant is a crop plant. Crop plants are all plants, used in agriculture. Accordingly in one embodiment the plant is a monocotyledonous plant, preferably a plant of the family Poaceae, Musaceae, Liliaceae or Bromeliaceae, preferably of the family Poaceae. Accordingly, in yet another embodiment the plant is a Poaceae plant of the genus Zea, Triticum, Oryza, Hordeum, Secale, Avena, Saccharum, Sorghum, Pennisetum, Setaria, Panicum, Eleusine, Miscanthus, Brachypodium, Festuca or Lolium. When the plant is of the genus Zea, the preferred species is Z. mays. When the plant is of the genus Triticum, the preferred species is T. aestivum, T. speltae or T. durum. When the plant is of the genus Oryza, the preferred species is O. sativa. When the plant is of the genus Hordeum, the preferred species is H. vulgare. When the plant is of the genus Secale, the preferred species S. cereale. When the plant is of the genus Avena, the preferred species is A. sativa. When the plant is of the genus Saccarum, the preferred species is S. officinarum. When the plant is of the genus Sorghum, the preferred species is S. vulgare, S. bicolor or S. sudanense. When the plant is of the genus Pennisetum, the preferred species is P. glaucum. When the plant is of the genus Setaria, the preferred species is S. italica. When the plant is of the genus Panicum, the preferred species is P. miliaceum or P. virgatum. When the plant is of the genus Eleusine, the preferred species is E. coracana. When the plant is of the genus Miscanthus, the preferred species is M. sinensis. When the plant is a plant of the genus Festuca, the preferred species is F. arundinaria, F. rubra or F. pratensis. When the plant is of the genus Lolium, the preferred species is L. perenne or L. multiflorum. Alternatively, the plant may be Triticosecale.

Alternatively, in one embodiment the plant is a dicotyledonous plant, preferably a plant of the family Fabaceae, Solanaceae, Brassicaceae, Chenopodiaceae, Asteraceae, Malvaceae, Linacea, Euphorbiaceae, Convolvulaceae Rosaceae, Cucurbitaceae, Theaceae, Rubiaceae, Sterculiaceae or Citrus. In one embodiment the plant is a plant of the family Fabaceae, Solanaceae or Brassicaceae. Accordingly, in one embodiment the plant is of the family Fabaceae, preferably of the genus Glycine, Pisum, Arachis, Cicer, Vicia, Phaseolus, Lupinus, Medicago or Lens. Preferred species of the family Fabaceae are M. truncatula, M, sativa, G. max, P. sativum, A. hypogea, C. arietinum, V. faba, P. vulgaris, Lupinus albus, Lupinus luteus, Lupinus angustifolius or Lens culinaris. More preferred are the species G. max A. hypogea and M. sativa. Most preferred is the species G. max. When the plant is of the family Solanaceae, the preferred genus is Solanum, Lycopersicon, Nicotiana or Capsicum. Preferred species of the family Solanaceae are S. tuberosum, L. esculentum, N. tabaccum or C. chinense. More preferred is S. tuberosum. Accordingly, in one embodiment the plant is of the family Brassicaceae, preferably of the genus Brassica or Raphanus. Preferred species of the family Brassicaceae are the species B. napus, B. oleracea, B. juncea or B. rapa. More preferred is the species B. napus. When the plant is of the family Chenopodiaceae, the preferred genus is Beta and the preferred species is the B. vulgaris. When the plant is of the family Asteraceae, the preferred genus is Helianthus and the preferred species is H. annuus. When the plant is of the family Malvaceae, the preferred genus is Gossypium or Abelmoschus. When the genus is Gossypium, the preferred species is G. hirsutum or G. barbadense and the most preferred species is G. hirsutum. A preferred species of the genus Abelmoschus is the species A. esculentus. When the plant is of the family Linacea, the preferred genus is Linum and the preferred species is L. usitatissimum. When the plant is of the family Euphorbiaceae, the preferred genus is Manihot, Jatropa or Rhizinus and the preferred species are M. esculenta, J. curcas or R. comunis. When the plant is of the family Convolvulaceae, the preferred genus is Ipomea and the preferred species is I. batatas. When the plant is of the family Rosaceae, the preferred genus is Rosa, Malus, Pyrus, Prunus, Rubus, Ribes, Vaccinium or Fragaria and the preferred species is the hybrid Fragaria×ananassa. When the plant is of the family Cucurbitaceae, the preferred genus is Cucumis, Citrullus or Cucurbita and the preferred species is Cucumis sativus, Citrullus lanatus or Cucurbita pepo. When the plant is of the family Theaceae, the preferred genus is Camellia and the preferred species is C. sinensis. When the plant is of the family Rubiaceae, the preferred genus is Coffea and the preferred species is C. arabica or C. canephora. When the plant is of the family Sterculiaceae, the preferred genus is Theobroma and the preferred species is T. cacao. When the plant is of the genus Citrus, the preferred species is C. sinensis, C. limon, C. reticulata, C. maxima and hybrids of Citrus species, or the like. In a preferred embodiment of the invention, the plant is a soybean, a potato or a corn plant. In one embodiment the plant is a Fabaceae plant and the target gene is substantially similar to SEQ ID NO: 1, 2, 19 or 29. In a further embodiment the plant is a Brassicaceae plant and the target gene is substantially identical to SEQ ID NO: 9. In an alternative embodiment the plant is a Solanaceae plant and the target gene is substantially identical to SEQ ID NO: 7 or 21. In a further embodiment the plant is a Poaceae plant and the target gene is substantially identical to SEQ ID NO: 13, 15 or 17. In one embodiment the plant is a Malvaceae plant and the target gene is substantially identical to SEQ ID NO: 25.

Suitable methods for transforming or transfecting host cells including plant cells are well known in the art of plant biotechnology. Any method may be used to transform the recombinant expression vector into plant cells to yield the transgenic plants of the invention. General methods for transforming dicotyledenous plants are disclosed, for example, in U.S. Pat. Nos. 4,940,838; 5,464,763, and the like. Methods for transforming specific dicotyledenous plants, for example, cotton, are set forth in U.S. Pat. Nos. 5,004,863; 5,159,135; and 5,846,797. Soybean transformation methods are set forth in U.S. Pat. Nos. 4,992,375; 5,416,011; 5,569,834; 5,824,877; 6,384,301 and in EP 0301749B1 may be used.

Transformation methods may include direct and indirect methods of transformation. Suitable direct methods include polyethylene glycol induced DNA uptake, liposome-mediated transformation (U.S. Pat. No. 4,536,475), biolistic methods using the gene gun (Fromm ME et al., Bio/Technology. 8(9):833-9, 1990; Gordon-Kamm et al. Plant Cell 2:603, 1990), electroporation, incubation of dry embryos in DNA-comprising solution, and microinjection. In the case of these direct transformation methods, the plasmids used need not meet any particular requirements. Simple plasmids, such as those of the pUC series, pBR322, M13mp series, pACYC184 and the like can be used. If intact plants are to be regenerated from the transformed cells, an additional selectable marker gene is preferably located on the plasmid. The direct transformation techniques are equally suitable for dicotyledonous and monocotyledonous plants.

Transformation can also be carried out by bacterial infection by means of Agrobacterium (for example EP 0 116 718), viral infection by means of viral vectors (EP 0 067 553; U.S. Pat. No. 4,407,956; WO 95/34668; WO 93/03161) or by means of pollen (EP 0 270 356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch R B et al. (1985) Science 225:1229. The Agrobacterium-mediated transformation is best suited to dicotyledonous plants but has also been adapted to monocotyledonous plants. The transformation of plants by Agro-bacteria is described in, for example, White F F, Vectors for Gene Transfer in Higher Plants, Transgenic Plants, Vol. 1, Engineering and Utilization, edited by S. D. Kung and R. Wu, Academic Press, 1993, pp. 15 -38; Jenes B et al. Techniques for Gene Transfer, Transgenic Plants, Vol. 1, Engineering and Utilization, edited by S. D. Kung and R. Wu, Academic Press, 1993, pp. 128-143; Potrykus (1991) Annu Rev Plant Physiol Plant Molec Biol 42:205-225.

Transformation may result in transient or stable transformation and expression. Although a nucleotide sequence of the present invention can be inserted into any plant and plant cell falling within these broad classes, it is particularly useful in crop plant cells.

Various tissues are suitable as starting material (explant) for the Agrobacterium-mediated transformation process including but not limited to callus (U.S. Pat. No. 5,591,616; EP-Al 604 662), immature embryos (EP-Al 672 752), pollen (U.S. Pat. No. 54,929,300), shoot apex (U.S. Pat. No. 5,164,310), or in planta transformation (U.S. Pat. No. 5,994,624). The method and material described herein can be combined with virtually all Agrobacterium mediated transformation methods known in the art. The transgenic plants of the invention may be crossed with similar transgenic plants or with transgenic plants lacking the nucleic acids of the invention or with non-transgenic plants, using known methods of plant breeding, to prepare seeds. Further, the transgenic plant of the present invention may comprise, and/or be crossed to another transgenic plant that comprises one or more nucleic acids, thus creating a “stack” of transgenes in the plant and/or its progeny. The seed is then planted to obtain a crossed fertile transgenic plant comprising the nucleic acid of the invention. The crossed fertile transgenic plant may have the particular expression cassette inherited through a female parent or through a male parent. The second plant may be an inbred plant. The crossed fertile transgenic may be a hybrid. Also included within the present invention are seeds of any of these crossed fertile transgenic plants. The seeds of this invention can be harvested from fertile transgenic plants and be used to grow progeny generations of transformed plants of this invention including hybrid plant lines comprising the DNA construct.

“Gene stacking” can also be accomplished by transferring two or more genes into the cell nucleus by plant transformation. Multiple genes may be introduced into the cell nucleus during transformation either sequentially or in unison. Multiple genes in plants or target pathogen species can be down-regulated by gene silencing mechanisms, specifically RNAi, by using a single transgene targeting multiple linked partial sequences of interest. Stacked, multiple genes under the control of individual promoters can also be over-expressed to attain a desired single or multiple phenotype. Constructs containing gene stacks of both over-expressed genes and silenced targets can also be introduced into plants yielding single or multiple agronomically important phenotypes. In certain embodiments the nucleic acid sequences of the present invention can be stacked with any combination of polynucleotide sequences of interest to create desired phenotypes. The combinations can produce plants with a variety of trait combinations including but not limited to disease resistance, herbicide tolerance, yield enhancement, cold and drought tolerance. These stacked combinations can be created by any method including but not limited to cross breeding plants by conventional methods or by genetic transformation. If the traits are stacked by genetic transformation, the polynucleotide sequences of interest can be combined sequentially or simultaneously in any order. For example if two genes are to be introduced, the two sequences can be contained in separate transformation cassettes or on the same transformation cassette. The expression of the sequences can be driven by the same or different promoters.

In accordance with this embodiment, the transgenic plant of the invention is produced by a method comprising the steps of providing an OPR3-like target gene, preparing an expression cassette having a first region that is substantially identical to a portion of the selected OPR3-like gene and a second region which is complementary to the first region, transforming the expression cassette into a plant, and selecting progeny of the transformed plant which express the dsRNA construct of the invention.

Increased resistance to nematode infection is a general trait wished to be inherited into a wide variety of plants. The present invention may be used to reduce crop destruction by any plant parasitic nematode. Preferably, the parasitic nematodes belong to nematode families inducing giant or syncytial cells. Nematodes inducing giant or syncytial cells are found in the families Longidoridae, Trichodoridae, Heterodidae, Meloidogynidae, Pratylenchidae or Tylenchulidae. In particular in the families Heterodidae and Meloidogynidae.

Accordingly, parasitic nematodes targeted by the present invention belong to one or more genus selected from the group of Naccobus, Cactodera, Dolichodera, Globodera, Heterodera, Punctodera, Longidorus or Meloidogyne. In a preferred embodiment the parasitic nematodes belong to one or more genus selected from the group of Naccobus, Cactodera, Dolichodera, Globodera, Heterodera, Punctodera or Meloidogyne. In a more preferred embodiment the parasitic nematodes belong to one or more genus selected from the group of Globodera, Heterodera, or Meloidogyne. In an even more preferred embodiment the parasitic nematodes belong to one or both genus selected from the group of Globodera or Heterodera. In another embodiment the parasitic nematodes belong to the genus Meloidogyne.

When the parasitic nematodes are of the genus Globodera, the species are preferably from the group consisting of G. achilleae, G. artemisiae, G. hypolysi, G. mexicana, G. millefolii, G. mali, G. pallida, G. rostochiensis, G. tabacum, and G. virginiae. In another preferred embodiment the parasitic Globodera nematodes includes at least one of the species G. pallida, G. tabacum, or G. rostochiensis. When the parasitic nematodes are of the genus Heterodera, the species may be preferably from the group consisting of H. avenae, H. carotae, H. ciceri, H. cruciferae, H. delvii, H. elachista, H. filipjevi, H. gambiensis, H. glycines, H. goettingiana, H. graduni, H. humuli, H. hordecalis, H. latipons, H. major, H. medicaginis, H. oryzicola, H. pakistanensis, H. rosii, H. sacchari, H. schachtii, H. sorghi, H. trifolii, H. urticae, H. vigni and H. zeae. In another preferred embodiment the parasitic Heterodera nematodes include at least one of the species H. glycines, H. avenae, H. cajani, H. gottingiana, H. trifolii, H. zeae or H. schachtii. In a more preferred embodiment the parasitic nematodes includes at least one of the species H. glycines or H. schachtii. In a most preferred embodiment the parasitic nematode is the species H. glycines.

When the parasitic nematodes are of the genus Meloidogyne, the parasitic nematode may be selected from the group consisting of M. acronea, M. arabica, M. arenaria, M. artiellia, M. brevicauda, M. camelliae, M. chitwoodi, M. cofeicola, M. esigua, M. graminicola, M. hapla, M. incognita, M. indica, M. inornata, M. javanica, M. lini, M. mali, M. microcephala, M. microtyla, M. naasi, M. salasi and M. thamesi. In a preferred embodiment the parasitic nematodes includes at least one of the species M. javanica, M. incognita, M. hapla, M. arenaria or M. chitwoodi.

The present invention also provides a method for inhibiting expression of a OPR3-like gene. In accordance with this embodiment, the method comprises the step of administering to the plant a dsRNA of the invention.

The following examples are not intended to limit the scope of the claims to the invention, but are rather intended to be exemplary of certain embodiments. Any variations in the exemplified methods that occur to the skilled artisan are intended to fall within the scope of the present invention.

### EXAMPLES

### Example 1

Cloning of OPR3-Like Gene from Soybean

Glycine max cv. Williams 82 was germinated and one day later, each seedling was inoculated with second stage juveniles (J2) of H. glycines race 3. Six days after inoculation, new root tissue was sliced into 1 cm long pieces, fixed, embedded in a cryomold, and sectioned using known methods. Syncytia cells were identified by their unique morphology of enlarged cell size, thickened cell wall, and dense cytoplasm and dissected into RNA extraction buffer using a PALM microscope (P.A.L.M. Microlaser Technologies GmbH, Bernried, Germany).

Total cellular RNA was extracted, amplified, and fluorescently labeled using known methods. As controls, total RNA was isolated from both “non-syncytia” and untreated control roots subjected to the same RNA amplification process. The amplified RNA was hybridized to proprietary soybean cDNA arrays.

Soybean cDNA clone 45174942 was identified as being up-regulated in syncytia of SCN-infected soybean roots. The 45174942 cDNA sequence (SEQ ID NO:1) was determined not to be full-length as there is no ATG start codon. The remaining residues were identified in the full length sequence (SEQ ID NO: 29) corresponding to 45174942 through 5′ RACE PCR as described in Example 4.

TABLE 2

| | Syncytia | Syncytia | Non- | Control |
|---|---|---|---|---|
| Gene Name | #1(N)^{¶} | #2(N) | Syncytia | Roots |
| 45174942^{§} | 311 ± 54(4) | 194 ± 46(5) | not detected | not detected |

### Example 2

### Generation Of Transgenic Soybean Roots And Nematode Bioassay

This exemplified method employs binary vectors containing the 45174942 target gene. The vector consists of a sense fragment (SEQ ID NO:2) of the target 45174942 gene, a spacer, antisense fragment of the target gene and a vector backbone. The target gene fragment (SEQ ID NO:2) corresponding to nucleotides 556 to 784 of SEQ ID NO:1 was used to construct the binary vectors RCB564, RCB573 and RCB582. In these vectors, dsRNA for the OPR3-like target gene was expressed under a syncytia or root preferred promoter, TPP promoter (SEQ ID NO: 4) in RCB564, A. thaliana promoter of locus At5g12170 (SEQ ID NO: 5), in RCB573 or MtN3-like promoter (SEQ ID NO: 6) in RCB582. These promoters drive transgene expression preferentially in roots and/or syncytia or giant cells. The selection marker for transformation was the mutated form of the acetohydroxy acid synthase (AHAS) selection gene (also referred to as AHAS2) from Arabidopsis thaliana (Sathasivan et al., Plant Phys. 97:1044-50, 1991), conferring resistance to the herbicide ARSENAL (imazapyr, BASF Corporation, Mount Olive, N.J.). The expression of AHAS2 was driven by a ubiquitin promoter from parsley (WO 03/102198).

### Example 3

### Rooted Explant Assays

The rooted explant assay was employed to demonstrate dsRNA expression and the resulting nematode resistance. This assay can be found in co-pending application Ser. No. 12/001,234, the contents of which are incorporated herein by reference.

Clean soybean seeds from soybean cultivar were surface sterilized and germinated. Three days before inoculation, an overnight liquid culture of the disarmed Agrobacterium culture, for example, the disarmed A. rhizogenes strain K599 containing the binary vector RCB564,

RCB573 or RCB582, was initiated. The next day the culture was spread onto an LB agar plate containing kanamycin as a selection agent. The plates were incubated at 28° C. for two days. One plate was prepared for every 50 explants to be inoculated. Cotyledons containing the proximal end from its connection with the seedlings were used as the explant for transformation. After removing the cotyledons the surface was scraped with a scalpel around the cut site. The cut and scraped cotyledon was the target for Agrobacterium inoculation. The prepared explants were dipped onto the disarmed thick A. rhizogenes colonies prepared above so that the colonies were visible on the cut and scraped surface. The explants were then placed onto 1% agar in Petri dishes for co-cultivation under light for 6-8 days.

After the transformation and co-cultivation, soybean explants were transferred to rooting induction medium with a selection agent, for example S-B5-708 for the mutated acetohydroxy acid synthase (AHAS) gene (Sathasivan et al., Plant Phys. 97:1044-50, 1991). Cultures were maintained in the same condition as in the co-cultivation step. The S-B5-708 medium comprises: 0.5X B5 salts, 3mM MES, 2% sucrose, 1×B5 vitamins, 400 μg/ml Timentin, 0.8% Noble agar, and 1 μM Imazapyr (selection agent for AHAS gene) (BASF Corporation, Florham Park, N.J.) at pH5.8.

Two to three weeks after the selection and root induction, transformed roots were formed on the cut ends of the explants. Explants were transferred to the same selection medium (S-B5-708 medium) for further selection. Transgenic roots proliferated well within one week in the medium and were ready to be subcultured.

Strong and white soybean roots were excised from the rooted explants and cultured in root growth medium supplemented with 200 mg/l Timentin (S-MS-606 medium) in six-well plates. Cultures were maintained at room temperature under the dark condition. The S-MS-606 medium comprises: 0.2× MS salts and B5 vitamins, 2% sucrose, and 200mg/l Timentin at pH5.8.

One to five days after sub-culturing, the roots were inoculated with surface sterilized nematode juveniles in multi-well plates for the gene of interest construct assay. As a control, soybean cultivar Williams 82 control vector and Jack control vector roots were used. The root cultures of each line that occupied at least half of the well were inoculated with surface-decontaminated race 3 of soybean cyst nematode (SCN) second stage juveniles (J2) at the level of 500 J2/well. The plates were then sealed and put back into the incubator at 25° C. in darkness. Several independent root lines were generated from each binary vector transformation and the lines were used for bioassay. Four weeks after nematode inoculation, the cysts in each well were counted. For each transformed line, the average number of cysts per line, the female index and the standard error values were determined across several replicated wells (Female index=average number of SCN cysts developing on the transgenic roots expressed as percentage of the average number of cysts developing on the W82 wild type susceptible control roots). Multiple independent, biologically replicated experiments were run for each expression construct. Rooted explant cultures transformed with constructs RCB564, RCB573 and RCB582 exhibited a general trend of reduced cyst numbers and female index relative to the known susceptible variety, Williams82.

### Example 4

### RACE PCR to Clone Full-Length 45174942 Coding Region

A full length transcript sequence with 100% homology to the partial cDNA clone 45174942 (SEQ ID NO: 1) was isolated using the GeneRacer Kit (L1502-01) from Invitrogen by following the manufacturers instructions. Total RNA from soybean roots harvested 6 days after infection with SCN was prepared according to the Invitrogen GeneRacer Kit protocol. The prepared RNA was reverse transcribed according to the GeneRacer Kit protocol and used as the RACE library template for PCR to isolate 5′ cDNA ends using primary and secondary (nested) PCR reactions according to the GeneRacer Kit protocol. Nested PCR reactions were performed according to manufacturer's instructions to obtain the desired amplification product.

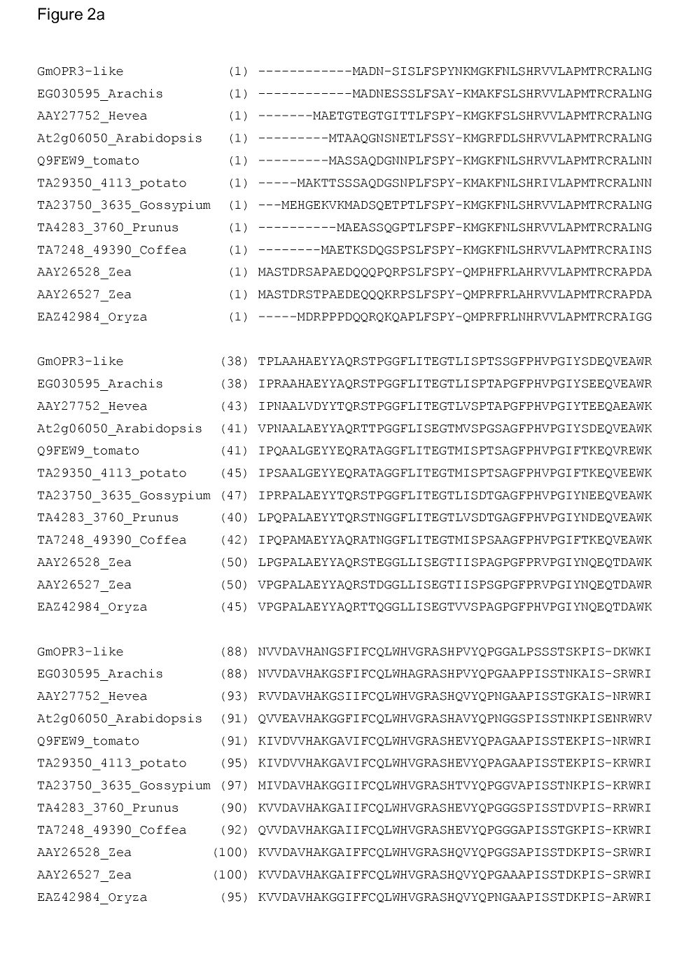

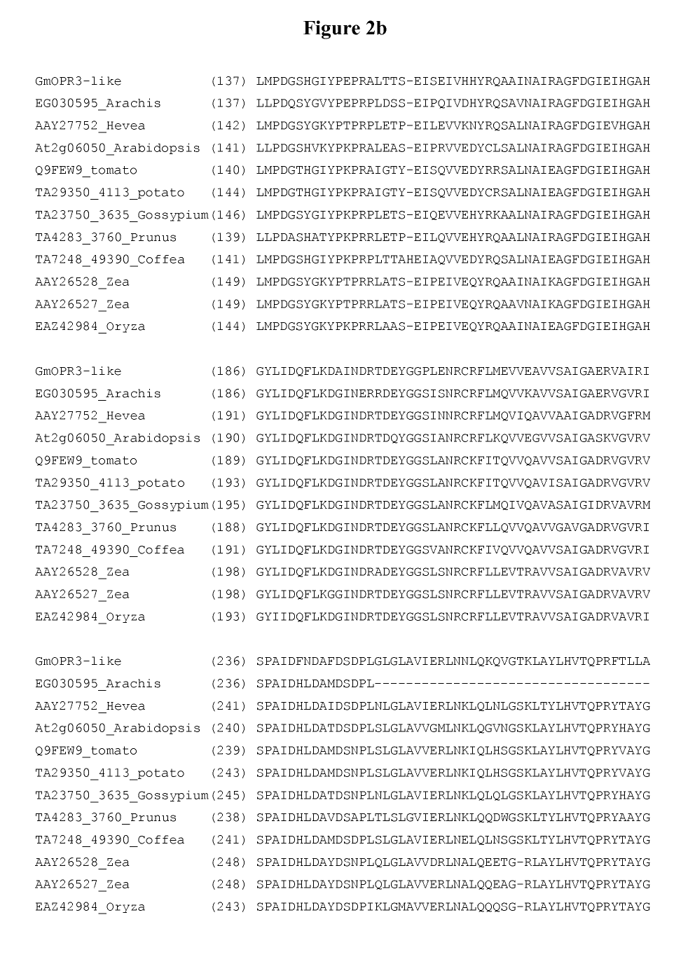

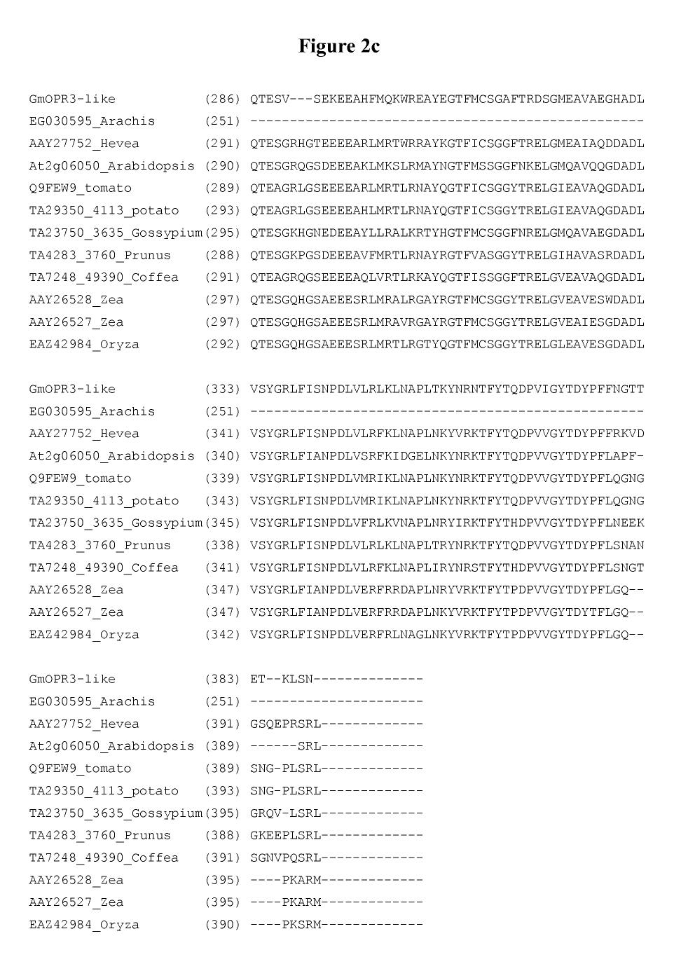

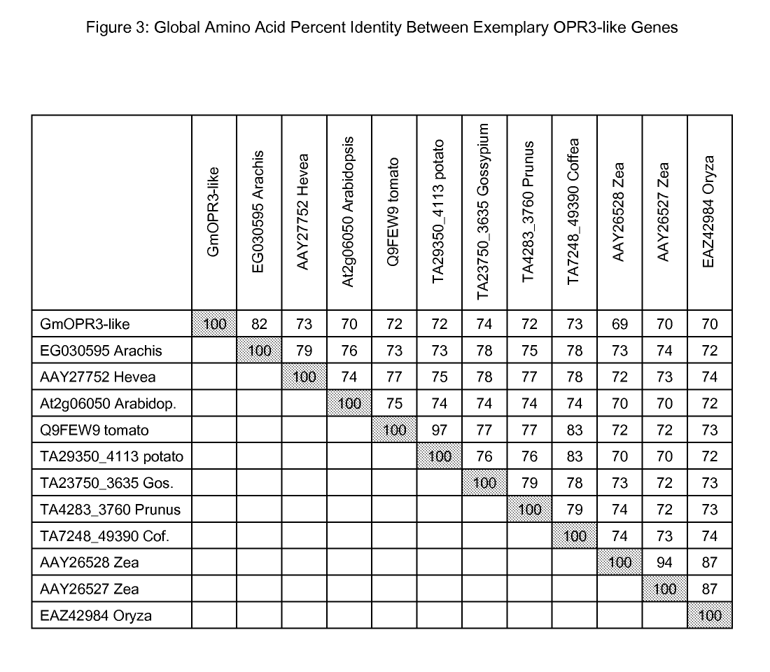

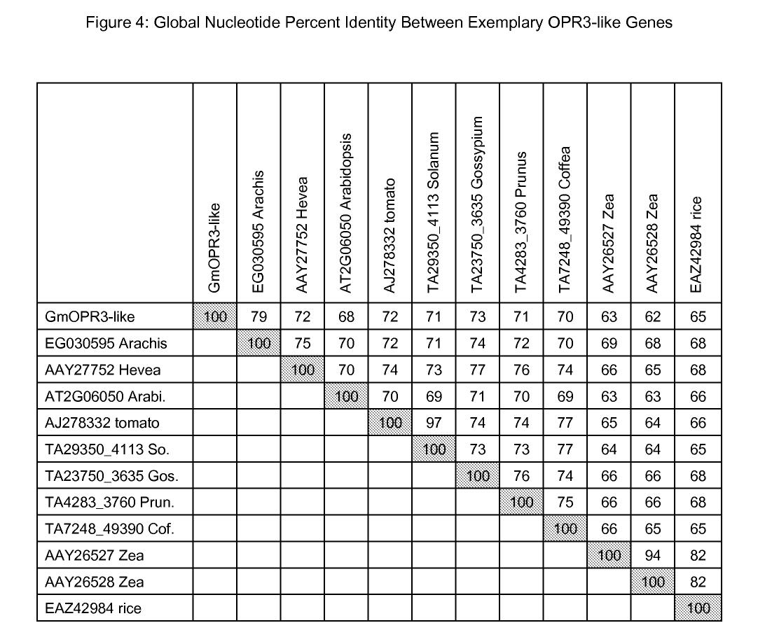

Specific products from secondary PCR reaction were separated by gel electrophoresis. Fragments were purified from agarose gel and cloned into pCR4-TOPO vectors (Invitrogen) following manufacturers instructions. Resulting colonies were miniprepped and sequenced. One of the full length fragments described as SEQ ID NO:29 (Full length GmOPR3-like DNA) had 100% percent identity with the overlapping region of SEQ ID NO:1 (45174942 DNA sequence). The alignment between proteins encoded by the full length Glycine max GmOPR3-like sequence and OPR3-like genes from other plant species is shown in FIGS. 2a-2c.

Those skilled in the art will recognize, or will be able to ascertain using no more than routine experimentation, many equivalents to the specific embodiments of the invention described herein. Such equivalents are intended to be encompassed by the following claims.

1. A dsRNA molecule comprising i) a first strand comprising a sequence substantially identical to a portion of a OPR3-like gene, and ii) a second strand comprising a sequence substantially complementary to the first strand, wherein the portion of the OPR3-like gene is from a polynucleotide selected from the group consisting of:
a) a polynucleotide comprising a sequence as set forth in SEQ ID NO:1, 2, 7, 9, 11, 13, 15, 17, 19, 21, 23, 25, 27, or 29;
b) a polynucleotide encoding a polypeptide having a sequence as set forth in SEQ ID NO:3, 8, 10, 12, 14, 16, 18, 20, 22, 24, 26, 28, or 30;
c) a polynucleotide having at least 70% sequence identity to a polynucleotide having a sequence as set forth in SEQ ID NO:1, 2, 7, 9, 11, 13, 15, 17, 19, 21, 23, 25, 27, or 29;
d) a polynucleotide encoding a polypeptide having at least 70% sequence identity to a polypeptide having a sequence as set forth in SEQ ID NO:3, 8, 10, 12, 14, 16, 18, 20, 22, 24, 26, 28, or 30;
e) a polynucleotide comprising a fragment of at least 19 consecutive nucleotides, or at least 50 consecutive nucleotides, or at least 100 consecutive nucleotides, or at least 200 consecutive nucleotides of a polynucleotide having a sequence as set forth in SEQ ID NO:1, 2, 7, 9, 11, 13, 15, 17, 19, 21, 23, 25, 27, or 29;
f) a polynucleotide comprising a fragment encoding a biologically active portion of a polypeptide having a sequence as set forth in SEQ ID NO:3, 8, 10, 12, 14, 16, 18, 20, 22, 24, 26, 28, or 30;
g) a polynucleotide hybridizing under stringent conditions to a polynucleotide comprising at least 19 consecutive nucleotides, or at least 50 consecutive nucleotides, or at least 100 consecutive nucleotides, or at least 200 consecutive nucleotides of a polynucleotide having a sequence as set forth in SEQ ID NO:1, 2, 7, 9, 11, 13, 15, 17, 19, 21, 23, 25, 27, or 29; and
h) a polynucleotide hybridizing under stringent conditions to a polynucleotide comprising at least 19 consecutive nucleotides, or at least 50 consecutive nucleotides, or at least 100 consecutive nucleotides, or at least 200 consecutive nucleotides of a polynucleotide encoding a polypeptide having a sequence as set forth in SEQ ID NO:3, 8, 10, 12, 14, 16, 18, 20, 22, 24, 26, 28, or 30.

2. The dsRNA of claim 1, wherein the polynucleotide comprises a sequence as set forth in SEQ ID NO:1, 2, 7, 9, 11, 13, 15, 17, 19, 21, 23, 25, 27, or 29.

3. The dsRNA of claim 1, wherein the polynucleotide has at least 70% sequence identity to a polynucleotide having a sequence as set forth in SEQ ID NO:1, 2, 7, 9, 11, 13, 15, 17, 19, 21, 23, 25, 27, or 29.

4. The dsRNA of claim 1, wherein the polynucleotide encodes a polypeptide having a sequence as set forth in SEQ ID NO:3, 8, 10, 12, 14, 16, 18, 20, 22, 24, 26, 28, or 30.

5. The dsRNA of claim 1, wherein the polynucleotide encodes a polypeptide having at least 70% sequence identity to a polypeptide having a sequence as set forth in SEQ ID NO:3, 8, 10, 12, 14, 16, 18, 20, 22, 24, 26, 28, or 30.

6. The dsRNA of claim 1, wherein the a polynucleotide hybridizes under stringent conditions to a polynucleotide comprising a sequence as set forth in SEQ ID NO:1, 2, 7, 9, 11, 13, 15, 17, 19, 21, 23, 25, 27, or 29.

7. The dsRNA of claim 1, wherein the polynucleotide comprises a fragment encoding a biologically active portion of a polypeptide having a sequence as set forth in SEQ ID NO:3, 8, 10, 12, 14, 16, 18, 20, 22, 24, 26, 28, or 30.

8. A pool of dsRNA molecules comprising a multiplicity of RNA molecules each comprising a double stranded region having a length of about 19 to 24 nucleotides, wherein said dsRNA molecules are derived from a portion of a polynucleotide selected from the group consisting of:
a) a polynucleotide comprising a sequence as set forth in SEQ ID NO:1, 2, 7, 9, 11, 13, 15, 17, 19, 21, 23, 25, 27, or 29;
b) a polynucleotide encoding a polypeptide having a sequence as set forth in SEQ ID NO:3, 8, 10, 12, 14, 16, 18, 20, 22, 24, 26, 28, or 30;
c) a polynucleotide having at least 90% sequence identity to a polynucleotide having a sequence as set forth in SEQ ID NO:1, 2, 7, 9, 11, 13, 15, 17, 19, 21, 23, 25, 27, or 29; and
d) a polynucleotide encoding a polypeptide having 90% sequence identity to a polypeptide having a sequence as set forth in SEQ ID NO:3, 8, 10, 12, 14, 16, 18, 20, 22, 24, 26, 28, or 30.

9. The pool of dsRNA of claim 8, wherein the polynucleotide comprises a sequence as set forth in SEQ ID NO:1, 2, 7, 9, 11, 13, 15, 17, 19, 21, 23, 25, 27, or 29.

10. The pool of dsRNA of claim 8, wherein the polynucleotide has at least 90% sequence identity to a polynucleotide having a sequence as set forth in SEQ ID NO:1, 2, 7, 9, 11, 13, 15, 17, 19, 21, 23, 25, 27, or 29.

11. The pool of dsRNA of claim 8, wherein the polynucleotide encodes a polypeptide having a sequence as set forth in SEQ ID NO:3, 8, 10, 12, 14, 16, 18, 20, 22, 24, 26, 28, or 30.

12. The pool of dsRNA of claim 8, wherein the polynucleotide encodes a polypeptide having at leat 90% sequence identity to a polypeptide having a sequence as set forth in SEQ ID NO:3, 8, 10, 12, 14, 16, 18, 20, 22, 24, 26, 28, or 30.

13. A transgenic plant capable of expressing a dsRNA that is substantially identical to a portion of an OPR3-like gene, wherein the portion of the OPR3-like gene is from a polynucleotide selected from the group consisting of:
a) a polynucleotide comprising a sequence as set forth in SEQ ID NO:1, 2, 7, 9, 11, 13, 15, 17, 19, 21, 23, 25, 27, or 29;
b) a polynucleotide encoding a polypeptide having a sequence as set forth in SEQ ID NO:3, 8, 10, 12, 14, 16, 18, 20, 22, 24, 26, 28, or 30;
c) a polynucleotide having at least 70% sequence identity to a polynucleotide having a sequence as set forth in SEQ ID NO:1, 2, 7, 9, 11, 13, 15, 17, 19, 21, 23, 25, 27, or 29;
d) a polynucleotide encoding a polypeptide having at least 70% sequence identity to a polypeptide having a sequence as set forth in SEQ ID NO:3, 8, 10, 12, 14, 16, 18, 20, 22, 24, 26, 28, or 30;
e) a polynucleotide comprising a fragment of at least 19 consecutive nucleotides, or at least 50 consecutive nucleotides, or at least 100 consecutive nucleotides, or at least 200 consecutive nucleotides of a polynucleotide having a sequence as set forth in SEQ ID NO:1, 2, 7, 9, 11, 13, 15, 17, 19, 21, 23, 25, 27, or 29;
f) a polynucleotide comprising a fragment encoding a biologically active portion of a polypeptide having a sequence as set forth in SEQ ID NO:3, 8, 10, 12, 14, 16, 18, 20, 22, 24, 26, 28, or 30;
g) a polynucleotide hybridizing under stringent conditions to a polynucleotide comprising at least 19 consecutive nucleotides, or at least 50 consecutive nucleotides, or at least 100 consecutive nucleotides, or at least 200 consecutive nucleotides of a polynucleotide having a sequence as set forth in SEQ ID NO:1, 2, 7, 9, 11, 13, 15, 17, 19, 21, 23, 25, 27, or 29; and
h) a polynucleotide hybridizing under stringent conditions to a polynucleotide comprising at least 19 consecutive nucleotides, or at least 50 consecutive nucleotides, or at least 100 consecutive nucleotides, or at least 200 consecutive nucleotides of a polynucleotide encoding a polypeptide having a sequence as set forth in SEQ ID NO:3, 8, 10, 12, 14, 16, 18, 20, 22, 24, 26, 28, or 30.

14. The transgenic plant of claim 13, wherein the dsRNA comprises a multiplicity of RNA molecules each comprising a double stranded region having a length of about 19 to 24 nucleotides, wherein said RNA molecules are derived from a portion of a polynucleotide selected from the group consisting of:
a) a polynucleotide comprising a sequence as set forth in SEQ ID NO:1, 2, 7, 9, 11, 13, 15, 17, 19, 21, 23, 25, 27, or 29;
b) a polynucleotide encoding a polypeptide having a sequence as set forth in SEQ ID NO:3, 8, 10, 12, 14, 16, 18, 20, 22, 24, 26, 28, or 30;
c) a polynucleotide having at least 90% sequence identity to a polynucleotide having a sequence as set forth in SEQ ID NO:1, 2, 7, 9, 11, 13, 15, 17, 19, 21, 23, 25, 27, or 29; and
d) a polynucleotide encoding a polypeptide having at least 90% sequence identity to a polypeptide having a sequence as set forth in SEQ ID NO:3, 8, 10, 12, 14, 16, 18, 20, 22, 24, 26, 28, or 30.

15. The transgenic plant of claim 13, wherein the plant is selected from the group consisting of maize, wheat, barley, sorghum, rye, triticale, rice, sugarcane, citrus trees, pineapple, coconut, banana, coffee, tea, tobacco, sunflower, pea, alfalfa, soybean, carrot, celery, tomato, potato, cotton, tobacco, eggplant, pepper, oilseed rape, canola, beet, cabbage, cauliflower, broccoli, lettuce, Lotus sp., Medicago truncatula, prerennial grass, ryegrass, and Arabidopsis thaliana.

16. The transgenic plant of claim 13, wherein the plant is soybean.

17. The transgenic plant of claim 13, wherein the polynucleotide comprises a sequence as set forth in SEQ ID NO:1, 2, 7, 9, 11, 13, 15, 17, 19, 21, 23, 25, 27, or 29.

18. The transgenic plant of claim 13, wherein the polynucleotide has at least 70% sequence identity to a polynucleotide having a sequence as set forth in SEQ ID NO:1, 2, 7, 9, 11, 13, 15, 17, 19, 21, 23, 25, 27, or 29.

19. The transgenic plant of claim 13, wherein the polynucleotide encodes a polypeptide having a sequence as set forth in SEQ ID NO:3, 8, 10, 12, 14, 16, 18, 20, 22, 24, 26, 28, or 30.

20. The transgenic plant of claim 13, wherein the polynucleotide encodes a polypeptide having at least 70% sequence identity to a polypeptide having a sequence as set forth in SEQ ID NO:3, 8, 10, 12, 14, 16, 18, 20, 22, 24, 26, 28, or 30.

21. A method of making a transgenic plant capable of expressing a dsRNA that inhibits expression of an OPR3-like target gene in the plant, said method comprises the steps of i) preparing a nucleic acid having a region that is substantially identical to a portion of the OPR3-like gene, wherein the nucleic acid is able to form a double-stranded transcript once expressed in the plant; ii) transforming a recipient plant with said nucleic acid; iii) producing one or more transgenic offspring of said recipient plant; and iv) selecting the offspring for expression of said transcript,
wherein the portion of the target gene is from 19 to 500 nucleotides of a polynucleotide selected from the group consisting of:
a) a polynucleotide comprising a sequence as set forth in SEQ ID NO:1, 2, 7, 9, 11, 13, 15, 17, 19, 21, 23, 25, 27, or 29;
b) a polynucleotide encoding a polypeptide having a sequence as set forth in SEQ ID NO:3, 8, 10, 12, 14, 16, 18, 20, 22, 24, 26, 28, or 30;
c) a polynucleotide having at least 70% sequence identity to a polynucleotide having a sequence as set forth in SEQ ID NO:1, 2, 7, 9, 11, 13, 15, 17, 19, 21, 23, 25, 27, or 29;
d) a polynucleotide encoding a polypeptide having at least 70% sequence identity to a polypeptide having a sequence as set forth in SEQ ID NO:3, 8, 10, 12, 14, 16, 18, 20, 22, 24, 26, 28, or 30;
e) a polynucleotide comprising a fragment of at least 19 consecutive nucleotides, or at least 50 consecutive nucleotides, or at least 100 consecutive nucleotides, or at least 200 consecutive nucleotides of a polynucleotide having a sequence as set forth in SEQ ID NO:1, 2, 7, 9, 11, 13, 15, 17, 19, 21, 23, 25, 27, or 29;
f) a polynucleotide comprising a fragment encoding a biologically active portion of a polypeptide having a sequence as set forth in SEQ ID NO:3, 8, 10, 12, 14, 16, 18, 20, 22, 24, 26, 28, or 30;
g) a polynucleotide hybridizing under stringent conditions to a polynucleotide comprising at least 19 consecutive nucleotides, or at least 50 consecutive nucleotides, or at least 100 consecutive nucleotides, or at least 200 consecutive nucleotides of a polynucleotide having a sequence as set forth in SEQ ID NO:1, 2, 7, 9, 11, 13, 15, 17, 19, 21, 23, 25, 27, or 29; and
h) a polynucleotide hybridizing under stringent conditions to a polynucleotide comprising at least 19 consecutive nucleotides, or at least 50 consecutive nucleotides, or at least 100 consecutive nucleotides, or at least 200 consecutive nucleotides of a polynucleotide encoding a polypeptide having a sequence as set forth in SEQ ID NO:3, 8, 10, 12, 14, 16, 18, 20, 22, 24, 26, 28, or 30.

22. The method of claim 21, wherein the dsRNA comprises a multiplicity of RNA molecules each comprising a double stranded region having a length of about 19 to 24 nucleotides, wherein said RNA molecules are derived from a polynucleotide selected from the group consisting of:
a) a polynucleotide comprising a sequence as set forth in SEQ ID NO:1, 2, 7, 9, 11, 13, 15, 17, 19, 21, 23, 25, 27, or 29;
b) a polynucleotide encoding a polypeptide having a sequence as set forth in SEQ ID NO:3, 8, 10, 12, 14, 16, 18, 20, 22, 24, 26, 28, or 30;
c) a polynucleotide having at least 90% sequence identity to a polynucleotide having a sequence as set forth in SEQ ID NO:1, 2, 7, 9, 11, 13, 15, 17, 19, 21, 23, 25, 27, or 29; and
d) a polynucleotide encoding a polypeptide having at least 90% sequence identity to a polypeptide having a sequence as set forth in SEQ ID NO:3, 8, 10, 12, 14, 16, 18, 20, 22, 24, 26, 28, or 30.

23. The method of claim 21, wherein the plant is selected from the group consisting of soybean, potato, tomato, peanuts, cotton, cassava, coffee, coconut, pineapple, citrus trees, banana, corn, rape, beet, sunflower, sorghum, wheat, oats, rye, barley, rice, green bean, lima bean, pea, and tobacco.

24. The method of claim 21, wherein the plant is soybean.

25. The method of claim 21, wherein the dsRNA is expressed in plant roots or nema-tode feeding sites.


SEQUENCE LISTING (30 sequences)

SEQ ID NO: 1 - DNA, 1410 bp, Glycine max
cagataactcaattagcttattttctccatacaacaagatgggcaaattcaacctctctcatagggtggtattggctcccatgaccagatgcagagcgctcaatgggactccactggcagcacatgctgaatactacgctcagagatcaacaccgggtggatttctcatcactgaaggcaccttgatctctccaacttcttctgggtttcctcatgttcctggaatatactcagatgaacaggtagaggcatggagaaatgtagtggacgccgtgcatgccaacggcagctttatcttctgtcaactctggcatgttggccgtgcatcacatccagtgtatcagcctggtggggctctaccctcttcgtccaccagcaaacccatatcagacaagtggaaaattctcatgcccgatggctcccatggcatctatccagagcctcgtgcacttaccacttctgagatatctgaaatagtgcatcattatcgccaagcagctattaatgcaattcgagcaggttttgatggaatcgagattcatggagcacatgggtatctcattgatcaattcttaaaggatgcaatcaatgatagaacagatgaatacggtggaccactagaaaaccggtgcaggttcttaatggaggtagttgaagctgttgtctctgccattggagcggaaagagttgctatcagaatttcaccagcaattgatttcaatgacgcctttgactctgacccacttgggctaggcttagcagtgattgaaagactcaacaatttgcagaaacaagtgggcacaaaactcgcttatcttcatgttactcagcctcgattcacacttttggcgcaaaccgagtcagtgagtgaaaaggaggaagctcatttcatgcagaaatggagagaggcttatgagggaacattcatgtgtagtggagcttttactagggactcaggaatggaagctgtagctgaaggccatgctgatttggtatcctatggtcgtcttttcatctccaatccagacttggttttaaggcttaagctcaatgcacctcttaccaagtataacaggaacacattttacacccaagatcctgttataggctacacagattatcctttctttaatggaacaactgagacaaaattaagtaactagctaaggccatgcatgccctttaattttaatctccatatggctttttgaataataatgttcataacattcaaaactcttcagttgagtttatcctcagacaaacaaattaagtggttcattcacttgttagggtatttagatcttaggttaattagtctccggcattttgatttcatttcaatttgtattcagtctttcattttgaataaaataatattaagttttttgccttaaaaaaaaaaaaaaa

SEQ ID NO: 2 - DNA, 229 bp, Glycine max
atctcattgatcaattcttaaaggatgcaatcaatgatagaacagatgaatacggtggaccactagaaaaccggtgcaggttcttaatggaggtagttgaagctgttgtctctgccattggagcggaaagagttgctatcagaatttcaccagcaattgatttcaatgacgcctttgactctgacccacttgggctaggcttagcagtgattgaaagactcaacaattt

SEQ ID NO: 3 - protein, 386 aa, Glycine max
DNSISLFSPYNKMGKFNLSHRVVLAPMTRCRALNGTPLAAHAEYYAQRSTPGGFLITEGTLISPTSSGFPHVPGIYSDEQVEAWRNVVDAVHANGSFIFCQLWHVGRASHPVYQPGGALPSSSTSKPISDKWKILMPDGSHGIYPEPRALTTSEISEIVHHYRQAAINAIRAGFDGIEIHGAHGYLIDQFLKDAINDRTDEYGGPLENRCRFLMEVVEAVVSAIGAERVAIRISPAIDFNDAFDSDPLGLGLAVIERLNNLQKQVGTKLAYLHVTQPRFTLLAQTESVSEKEEAHFMQKWREAYEGTFMCSGAFTRDSGMEAVAEGHADLVSYGRLFISNPDLVLRLKLNAPLTKYNRNTFYTQDPVIGYTDYPFFNGTTETKLSN

SEQ ID NO: 4 - DNA, 1999 bp, Arabidopsis thaliana
gtagtgcccttcatggataccaaaagagaaaatttgatttagtgcatacatataacaatataacgccgcataataatactgtataaaacagtcatgtaacgatatgacagcagtaatacagttccaagagacgttataatcgtatgcaatcatatgcttgcgtagattttccaacagttttgtttcgttgataggaggaactcaacactctagggtagtgattggtagacactattagcacaaaaaatattaattttactctgatgtttaccaaaaaagttaccaatcaaatatttaagagatcgtactcttccacggcgactctaaaaaccaaagatataggttagactcataactactttataaagaaaatgtttaacgataactaccgagatctaataaataaaccttcattttcaagtatattatatttgcttcttttgtttatatatcaaaccaagttctggtttataaaaatattagataaaactcgtctaaataggtaggtgtaaaataaaattttaaatttttatcgataatatttaaaatttgaaaagttaataatgatccacacattttttctaatatttaatttagtaatttttgtattaaataaaatttcaatcatatacattcgatttttctatacattttaactatctatttctgcataataaactgtattttcattttatacgcttcatcttatggatgatatttaaattttaaatagtaattcatacactttttaatatttaatttagtattttcttaaatccaaattttaatcttacaatttaaatatctactttaacataatacaaatacaatttaatttcattgtattaaattcaaatataatttgattataataaaatacaatttaattctaaaaagtccatcttagattttaattttcctttttagttttgaaaattaaaaatttaaatttattagatatatatgttactttttcagttttcctatttatttaagaaaaaaatattttttaacacatgtcaacttgtaaacaatagactgaacacgtcattttatattatgtttagttttgaaaattaaagttaattaaatatttatatttcttttttttagcttttctaattatttttaaaatagtaaatatttttaatacaaatcaatatctgaacaatagatttgatacataacataatcctataaattattaacttggaaaacgatagtttatataataaaattattttcttaagttctctaaccataacaattaaactatattttagcgaagaaaagaagagaataccgagagaacgcaacttgcactaaaagctaccactttggcaaatcactcatttatattattatatactatcacctcaattcaatcgaaacctcaaaataacactaatatatacacaaagaaacaacagaataacaccgaagaatataggtttaggaaaatccagaatttgttgagactaaagagatcaaattttcgatacaaggttttgctcaatttgtattttcataataaaattctttatttcaccatagacttacatgattagtttttcttttaataaaaaaaaacacgcgacatgaaaattatattatctcagtgttgtcgaatttgaatttgaattttgagttaaatactacacatttgttgacaacttattaaactttacaagtctgctacaaatattgtcaaatatttactaattaatggaccaaaatcctctaacttgcaaatttgtatctacatcaacttaaaaattaggaatatgcgacccaaaaaaaaaaaaactaggaataataataaaaaaatggaatgatgtggaggaagctctttactctttgagaggaagtttataaattgaccacacatttagtctattatcatcacatgtattaagacttgacaacttgtctttctcacaccaaacccctctcctctgtttcataacatctgctctttcttttttttcctaagccccta

SEQ ID NO: 5 - DNA, 1476 bp, Arabidopsis thaliana
gctcgcgttagttccactcaaggagtatcctttcttccttgcgcaactctccaccttcgggtaaagtaccatctctagcatcttgagtcttgatcaacttctgttttgcttactctcaaaatgcattaatttttttttatactagatcatagtattatatctcttaatctacctattgaaatctacttaatgtttttactaaaacctacgtgtttctctttagagaattttgtgctatgcatgaattagaggttagtaatgtgtaatacttcataagtctagatttatttgttggttaacacgtttagtaattcacacacacacaccaccttagatattttactgtgaattagaaaaagatacatagttaggagtgtttttttaaaaaaattcaatcatgagaaaattagaggtgtgatgttatacattatgaaaatgcaaagggcagatacgaataaattagaaacttgtttaacgggtcagagttggcttctagtctctttcgacttggatacttcttcttctacaattgggacattattgtaggcgcattatatcatttctctacatgcaatgaatgtacatacattaattcacatttatttttggaataatcatatgagtgatcgaagtttgtatttatatattcaatcttcacaaactacttttatttaaaaatcatttgcaaaatgctattttattgacaaaaagatatatgctataaaataaaataaaattcacaaactatagtcattaatacaaaaagaaatcattgaatatggtagaggggaaacaaaaaaaaaacacgacgatgtaagttggtggaaccacattatcaaaataaaagaaggtggtggaaccaaattgaataaagtccgtccatatcattatccgtcccttaggagcctctaattagtaatattcttatgggtccactgtggcttagaggacttgattaaaaccattcttatttagtgctaactttgtgagggttggaataacgaaccaagctgattcaaaccattccaaaacaaagttgtcacatatttcaaaaccaaagtttaccggacagagaaatatggtgtgtttttctcaaaccaagctaaatggaatccattgtaaaccaaaatgttcacacctacctattcttttggagtcccttttccatgtgtttgctgtctgctagtcaagtttcattagctgattgccttgcatcatattcttggatcaactttttttttttttttttttggggtaattaacaaaatgcttaaatttctcaagactataggatcacattacctgtgtgcttaacataacttttagataggctagagaattgatctattacaagataatcaataatttacagaagaaaacattcttttttttgttctatttccttcatgtaggtatgtagctgtatattatactatcttgtattttcgatatcgtgctggaactgtcacagatgca

SEQ ID NO: 6 - DNA, 609 bp, Glycine max
gaagccacgtcatgaagagtatatcatttcagtaatgttttgagacgcctctataatgctttaccaacaaaacaaaacaaaaaaaagaacatttgaaaccatttgtattaaaaaaaaaaaggtatattaggccataatattataggtaacatgaaatatcaaatgacacgcaagagttttgtcaaaaatgaaaccatcacacatcagagattatggcaaataatgttttgtgtgtctcttgcttcacccataacataagcctctataactggagagaagaaaaaaaaaagtggaggggctagggtgggaatttggaagaatacagttatattgagcattgagcaagttgatagaaagcttctcaatttgtacaaaatttgcatccacatgattattaaagacgtagacagcacttcttccttctttttttctataagtttcttatatattgttcttcatgttttaatattattactttatgtacgcgtctaacagtagtcctcccaaactgctataaatagagcctcttcaacgcacctcttggcagtacaaaaattattcatctcttctaagttctaattttctaagcattcagtaaaagaactaacc

SEQ ID NO: 7 - DNA, 1191 bp, Lycopersicon esculentum
atggcgtcttcagctcaagatggaaacaatccccttttctctccttacaagatgggcaagttcaatctatcccacagggtagtattggctccgatgacaaggtgcagagcactgaataatattccacaggcggcgctaggggagtattacgagcagagagcgacggccggtggatttctgatcactgaaggcactatgatttctccgacttcagctgggtttcctcatgtgccagggattttcacaaaggaacaagtaagggaatggaagaaaatagttgatgtagtgcatgcaaagggtgctgtcatattttgtcagctgtggcatgttggtcgtgcatctcatgaagtgtatcaacctgctggagctgcaccaatatcatccactgagaagcctatatcaaataggtggagaattctaatgcctgatggaactcatgggatttatccaaaaccaagagcaattggaacctatgagatctcacaagttgttgaagattatcgcaggtcggccttgaatgctattgaagcaggtttcgatggtattgaaatccatggagctcacggttacttgattgatcaattcttgaaagatgggatcaatgaccggacagatgagtatggtggatcactagccaaccggtgcaaattcatcacacaggtggttcaagcagtagtctcagcaataggagctgatcgcgtaggcgttagagtttcaccagcaatagatcatcttgatgccatggactctaatccactcagccttggcttagcagttgttgaaagactaaacaaaatccaactccattctggttccaagcttgcctatcttcatgtaacacagccacgatacgtagcatatgggcaaactgaagcaggcagacttggcagtgaagaggaagaggctcgtttaatgaggactttgaggaacgcgtatcaggggacattcatttgcagtggtggatacactagggaactaggaattgaggctgtggcacaaggtgatgctgatctcgtgtcatatggtcgtcttttcatctctaatcctgatttggttatgagaatcaagctaaatgcacctctaaataagtataacaggaagacattctatactcaagatccagttgtgggatacacagattaccctttccttcaaggaaatggaagcaatggaccgttatcgcgtctgtga

SEQ ID NO: 8 - protein, 396 aa, Lycopersicon esculentum
MASSAQDGNNPLFSPYKMGKFNLSHRVVLAPMTRCRALNNIPQAALGEYYEQRATAGGFLITEGTMISPTSAGFPHVPGIFTKEQVREWKKIVDVVHAKGAVIFCQLWHVGRASHEVYQPAGAAPISSTEKPISNRWRILMPDGTHGIYPKPRAIGTYEISQVVEDYRRSALNAIEAGFDGIEIHGAHGYLIDQFLKDGINDRTDEYGGSLANRCKFITQVVQAVVSAIGADRVGVRVSPAIDHLDAMDSNPLSLGLAVVERLNKIQLHSGSKLAYLHVTQPRYVAYGQTEAGRLGSEEEEARLMRTLRNAYQGTFICSGGYTRELGIEAVAQGDADLVSYGRLFISNPDLVMRIKLNAPLNKYNRKTFYTQDPVVGYTDYPFLQGNGSNGPLSRL

SEQ ID NO: 9 - DNA, 1176 bp, Arabidopsis thaliana
atgacggcggcacaagggaactctaacgagactctgttttcttcttacaagatgggaagattcgatctctctcatcgagtggttctggcgccgatgacgcggtgcagggcgttgaacggagtaccaaacgcggcgttggcagagtattatgctcaacggaccactcccggcggttttctcatctccgaaggcaccatggtctctcccggatccgcagggttcccacatgtgcctggaatctattcagatgaacaagtagaagcatggaagcaagttgtggaagcagttcacgctaagggaggtttcatcttttgtcaattatggcatgttggacgtgcttctcatgcagtgtatcaacctaatggaggatcaccaatatcgtcaacgaacaaaccaatctcggaaaacaggtggcgagttttgttgcccgatggttcccacgtgaagtacccgaaacctcgggctttagaagcttccgagatacctcgggtggtggaggattattgcctttctgctttgaatgcgattcgagctggtttcgatgggattgagatccacggggcgcatggttaccttattgatcagtttttgaaagatgggatcaatgaccgtactgaccaatacggaggatccattgcaaaccgttgtagattcttgaaacaagtagtggaaggtgtagtttcagccataggagctagtaaagttggtgtgagggtatctccggctatagatcacttggacgcaactgattctgacccactatcactcgggctagccgtggttggcatgctcaataagttacaaggtgttaatggctcaaagctcgcttaccttcacgttacacaacctcgctaccacgcctacgggcaaacagagtctggaaggcaagggagtgatgaggaagaagctaagctaatgaagagcttgaggatggcttataatggaacctttatgtctagtggaggattcaataaggaactaggtatgcaagctgttcaacaaggtgatgctgatttggtttcatatggcagactgtttatcgcaaacccggatttggtttcgcggttcaagattgatggagagttgaataaatataatcggaagacgttttacactcaagatccagttgttggctacacggattatcctttcttggctcctttttcccgcctctga

SEQ ID NO: 10 - protein, 391 aa, Arabidopsis thaliana
MTAAQGNSNETLFSSYKMGRFDLSHRVVLAPMTRCRALNGVPNAALAEYYAQRTTPGGFLISEGTMVSPGSAGFPHVPGIYSDEQVEAWKQVVEAVHAKGGFIFCQLWHVGRASHAVYQPNGGSPISSTNKPISENRWRVLLPDGSHVKYPKPRALEASEIPRVVEDYCLSALNAIRAGFDGIEIHGAHGYLIDQFLKDGINDRTDQYGGSIANRCRFLKQVVEGVVSAIGASKVGVRVSPAIDHLDATDSDPLSLGLAVVGMLNKLQGVNGSKLAYLHVTQPRYHAYGQTESGRQGSDEEEAKLMKSLRMAYNGTFMSSGGFNKELGMQAVQQGDADLVSYGRLFIANPDLVSRFKIDGELNKYNRKTFYTQDPVVGYTDYPFLAPFSRL

SEQ ID NO: 11 - DNA, 1200 bp, Hevea brasiliensis
atggctgaaactggaacagaagggaccgggatcaccaccctattttctccttacaaaatgggcaagttcagcctctcccacagggtggtgctggctcccatgactagatgcagagcgttgaacgggataccaaacgcagcgctggtggattactacacgcagagatcaactcccggcggatttctcatcacggaaggcactctggtttcccctactgcccctgggtttcctcatgtccctggaatttatacagaagaacaagcggaggcatggaagagggtggtggatgcagttcatgccaaagggagcatcatattctgtcaattatggcatgttggccgcgcatctcatcaggtttatcaacctaatggagctgcaccaatatcatcgacgggcaaggccatctcaaacagatggagaattctcatgccagatggatcatatgggaaatacccaacacctaggcccttggaaacacctgaaatactagaggtagtgaagaattatcgccagtcagccttgaatgccattcgagcaggctttgatggaattgaggtccacggggctcatggttaccttattgatcaattcttaaaagacgggatcaatgaccgaacagatgagtatggtggatcaatcaacaatcgatgcagattcctaatgcaggtgattcaggcagtagttgcagctattggtgctgatcgagttggtttcagaatgtcaccggcaattgatcacctagatgccatagattctgatccgctcaacttgggtcttgctgtaatcgagagacttaacaaacttcagttgaaccttggatcaaaactcacttatctccatgtcactcagcctcgctacacagcttatggccaaacagaatcaggcagacatggtactgaagaagaggaagctagattaatgagaacttggagaagggcttataagggaactttcatctgtagcggtgggttcacgagggagctaggaatggaagctatagctcaagatgatgcagatttggtatcttatggccgactttttatttcaaacccagacttagtcttgagatttaagctcaatgcgcccttgaataagtatgtcaggaaaacattctacacccaagatcctgttgttgggtacacagactacccatttttcagaaaagtagacgggagccaggagccacgatcacgcctttga

SEQ ID NO: 12 - protein, 399 aa, Hevea brasiliensis
MAETGTEGTGITTLFSPYKMGKFSLSHRVVLAPMTRCRALNGIPNAALVDYYTQRSTPGGFLITEGTLVSPTAPGFPHVPGIYTEEQAEAWKRVVDAVHAKGSIIFCQLWHVGRASHQVYQPNGAAPISSTGKAISNRWRILMPDGSYGKYPTPRPLETPEILEVVKNYRQSALNAIRAGFDGIEVHGAHGYLIDQFLKDGINDRTDEYGGSINNRCRFLMQVIQAVVAAIGADRVGFRMSPAIDHLDAIDSDPLNLGLAVIERLNKLQLNLGSKLTYLHVTQPRYTAYGQTESGRHGTEEEEARLMRTWRRAYKGTFICSGGFTRELGMEAIAQDDADLVSYGRLFISNPDLVLRFKLNAPLNKYVRKTFYTQDPVVGYTDYPFFRKVDGSQEPRSRL

SEQ ID NO: 13 - DNA, 1185 bp, Oryza sativa
atggatcggccgccgccggatcagcagcggcagaagcaggcgccgctcttctcgccgtaccagatgccccgcttccgcctcaaccaccgggtggtgctggcgccgatgacgcggtgcagggcgatcggcggggtgcccggcccggcgctggcggagtactacgctcagcggaccacccagggtggcctgctcatctccgagggcaccgtcgtctcgcccgctggcccggggtttcctcatgtccctgggatatacaatcaagagcagactgatgcatggaagaaggtggtggatgctgttcatgccaagggaggcatctttttctgccagttatggcatgtaggcagagcttctcaccaagtataccagccaaacggtgctgcaccaatatcctcaactgataagccaatatcagcaagatggagaatactgatgcctgatggctcctatggcaagtatcctaaacctaggcgcctggcagcatcggaaatacctgaaattgtcgaacaatatcgtcaagccgccattaatgccattgaagcaggttttgatggcattgagatccatggtgctcatggctatatcattgatcaattcctaaaggatggaatcaatgaccgcactgacgagtatggtggctcactttccaaccgctgccggttcctacttgaggtaactagggctgtggtttctgccattggagcagaccgagtcgcggtgaggatatcaccagccattgatcaccttgacgcctatgattcagaccccattaagctcggcatggccgttgttgagcggctgaatgctctccagcagcagtcagggcggctcgcctacctccacgtcacgcagccacggtacaccgcctacgggcagaccgagtctgggcagcatggcagtgccgaggaggagagccgcctgatgcgcaccctccggggcacgtaccagggcacattcatgtgcagtggcggctacacgcgggagcttgggttggaagcagtggagagcggcgatgccgacctggtgtcgtacgggcggctcttcatatcaaacccggacctggtcgagcggttcaggctgaacgccgggctgaacaagtacgtgcgcaagacattctacacgcccgatcctgtcgtgggttacacggactatccgttcctcggacagcctaagtcgcggatgtaa

SEQ ID NO: 14 - protein, 394 aa, Oryza sativa
MDRPPPDQQRQKQAPLFSPYQMPRFRLNHRVVLAPMTRCRAIGGVPGPALAEYYAQRTTQGGLLISEGTVVSPAGPGFPHVPGIYNQEQTDAWKKVVDAVHAKGGIFFCQLWHVGRASHQVYQPNGAAPISSTDKPISARWRILMPDGSYGKYPKPRRLAASEIPEIVEQYRQAAINAIEAGFDGIEIHGAHGYIIDQFLKDGINDRTDEYGGSLSNRCRFLLEVTRAVVSAIGADRVAVRISPAIDHLDAYDSDPIKLGMAVVERLNALQQQSGRLAYLHVTQPRYTAYGQTESGQHGSAEEESRLMRTLRGTYQGTFMCSGGYTRELGLEAVESGDADLVSYGRLFISNPDLVERFRLNAGLNKYVRKTFYTPDPVVGYTDYPFLGQPKSRM

SEQ ID NO: 15 - DNA, 1200 bp, Zea mays
atggcctccacggatcgctccacgccggcggaggacgagcaacagcagaagcgcccgtctctcttctcgccgtaccagatgccccgcttccgcctcgcccaccgggtggtgctggcgccgatgaccaggtgcagggcgcccgacgcggtcccaggccccgcgctcgcggagtactacgcgcagcggtccacggacggcggcttgctcatctccgagggcaccatcatctcgccgtccggccctgggttccctcgtgtccctgggatatacaatcaagaacagactgatgcatggagaaaggtggttgatgctgttcatgccaagggagctatctttttctgccaactatggcatgtaggccgagcttctcaccaagtatatcagccgggtgctgctgctccgatatcctcaactgataagccaatatcatcaagatggaggatactgatgcccgatggatcctatggcaagtatccaactccgaggcgcctagccacatccgagataccagaaattgttgagcaataccgtcaagcagccgtaaacgccatcaaagcaggtttcgatggcatcgagatccatggcgcccatggctacctcatcgatcagttcctcaagggcggtatcaacgaccggactgacgagtacggtggctcactctccaaccgttgccggttcctcctggaggtgacccgagccgtggtctctgcgataggggcagaccgcgtcgcggtccgagtgtccccggccatcgaccatctcgacgcctacgactccaaccccctgcagctcggcctggccgtggtggagcgtctcaacgctctccagcaggaggccgggcggctggcctacctccacgtgacgcagccacggtacacggcgtacgggcagacagagtctggccagcacgggagtgccgaggaggagagccggctgatgcgtgccgtgcgaggtgcctaccgtggcacgttcatgtgcagcggtgggtacacgcgggagctcggggtcgaggccatcgagtccggggacgctgacctggtgtcctacgggcggctgttcatcgctaatcccgacctggtggagcggtttcggcgcgacgccccgctgaacaaatacgtgcgcaagacgttctacacgccggaccccgtcgtcggttacacggactacacattcctcggccagcctaaggcacgcatgtga

SEQ ID NO: 16 - protein, 399 aa, Zea mays
MASTDRSTPAEDEQQQKRPSLFSPYQMPRFRLAHRVVLAPMTRCRAPDAVPGPALAEYYAQRSTDGGLLISEGTIISPSGPGFPRVPGIYNQEQTDAWRKVVDAVHAKGAIFFCQLWHVGRASHQVYQPGAAAPISSTDKPISSRWRILMPDGSYGKYPTPRRLATSEIPEIVEQYRQAAVNAIKAGFDGIEIHGAHGYLIDQFLKGGINDRTDEYGGSLSNRCRFLLEVTRAVVSAIGADRVAVRVSPAIDHLDAYDSNPLQLGLAVVERLNALQQEAGRLAYLHVTQPRYTAYGQTESGQHGSAEEESRLMRAVRGAYRGTFMCSGGYTRELGVEAIESGDADLVSYGRLFIANPDLVERFRRDAPLNKYVRKTFYTPDPVVGYTDYTFLGQPKARM

SEQ ID NO: 17 - DNA, 1200 bp, Zea mays
atggcctccacggatcgctccgcgccggcggaggaccagcaacagccgcagcgcccgtccctcttctcgccgtaccagatgccccacttccgcctcgcgcaccgggtggtgctggcgccgatgaccaggtgccgggcgcccgatgcgctcccgggccccgcgctcgcggagtactacgcgcagcggtccacggaaggcggcttgctcatctccgagggcaccatcatctcgcccgccggccctgggttccctcgtgtccctgggatatacaatcaagagcagactgatgcatggaaaaaggtggttgatgctgttcatgccaagggagccatctttttctgccaactatggcatgtaggtcgagcttctcaccaagtatatcagccgggtggttctgctccaatatcctctactgataaaccaatatcatcaagatggaggatactgatgcccgatggatcctatggcaagtatccaactccgaggcgcctagccacatccgagataccagaaattgtcgagcaataccgacaggctgccataaacgccatcaaagcaggtttcgatggcatcgagatccacggtgcccatggctacctaatcgaccagttcctcaaggacggtatcaacgacagggctgacgagtacggtggctcactctccaaccgctgccggttcctcctggaggtgacccgcgccgtggtctccgcgataggggcagaccgggtggcggtccgggtgtccccggccatcgaccacctcgacgcgtacgactccaacccgctgcagctcggcctggccgtagtggaccgcctcaacgctctccaggaggagaccgggcggctggcctacctgcacgtgacgcagccacggtacacggcgtacgggcagacggagtccggccagcacgggagcgccgaggaggagagccggctgatgcgcgccctgcggggcgcctaccgcggcacgttcatgtgcagcggtgggtacacgcgcgagctcggcgtggaggccgtcgagtcgtgggacgccgacctggtgtcctacgggcggctgttcatcgctaacccggacctggtggagcggttccggcgcgacgccccgctgaacagatacgtgcgcaagacgttctacaccccggatcccgtcgttggttacacggactacccgttcctcggccagcctaaggcgcgcatgtga

SEQ ID NO: 18 - protein, 399 aa, Zea mays
MASTDRSAPAEDQQQPQRPSLFSPYQMPHFRLAHRVVLAPMTRCRAPDALPGPALAEYYAQRSTEGGLLISEGTIISPAGPGFPRVPGIYNQEQTDAWKKVVDAVHAKGAIFFCQLWHVGRASHQVYQPGGSAPISSTDKPISSRWRILMPDGSYGKYPTPRRLATSEIPEIVEQYRQAAINAIKAGFDGIEIHGAHGYLIDQFLKDGINDRADEYGGSLSNRCRFLLEVTRAVVSAIGADRVAVRVSPAIDHLDAYDSNPLQLGLAVVDRLNALQEETGRLAYLHVTQPRYTAYGQTESGQHGSAEEESRLMRALRGAYRGTFMCSGGYTRELGVEAVESWDADLVSYGRLFIANPDLVERFRRDAPLNRYVRKTFYTPDPVVGYTDYPFLGQPKARM

SEQ ID NO: 19 - DNA, 762 bp, Arachis hypogaea
atggcagacaacgagtccagcagcctgttttctgcttacaagatggcaaaattcagtctctcgcacagggtggtgttggcgcccatgaccaggtgcagagccttgaacggcatcccacgtgccgctcacgcggagtattacgctcagagatccacacccggtggattcctcatcaccgaagggactttgatctctcccactgctcctggcttccctcatgtacctggaatatactctgaggagcaagttgaggcatggagaaacgtcgtggatgccgtgcatgccaaaggcagcttcatcttctgtcaactctggcatgctggccgtgcatctcatcccgtgtatcagcctggggcggcgccgcccatttcctccacaaacaaggctatttcctccagatggagaattctcttgccggatcagtcctacggcgtgtatccagagccccgaccacttgactcttctgagataccacaaatagtggaccactatcgccagtcagcggtcaacgctatccgagcaggtttcgatggaattgagattcacggtgcacacggctatctgattgatcaattcttgaaggacgggatcaatgagcgaagagatgagtatggtggatccatttcaaataggtgcaggttcttaatgcaggtagttaaagcagttgtttctgcaattggagcagaaagagtaggtgttagaatctcaccggcaatcgaccacctggatgccatggactccgacccgcttgcctggggctag

SEQ ID NO: 20 - protein, 250 aa, Arachis hypogaea
MADNESSSLFSAYKMAKFSLSHRVVLAPMTRCRALNGIPRAAHAEYYAQRSTPGGFLITEGTLISPTAPGFPHVPGIYSEEQVEAWRNVVDAVHAKGSFIFCQLWHAGRASHPVYQPGAAPPISSTNKAISSRWRILLPDQSYGVYPEPRPLDSSEIPQIVDHYRQSAVNAIRAGFDGIEIHGAHGYLIDQFLKDGINERRDEYGGSISNRCRFLMQVVKAVVSAIGAERVGVRISPAIDHLDAMDSDPL

SEQ ID NO: 21 - DNA, 1203 bp, Solanum tuberosum
atggctaaaacgacatcgtcttcagctcaagatggaagcaatcccctcttctctccttacaagatggcaaagttcaatctatcccacaggatagtattggctccgatgacaaggtgcagagcattgaataatattccttcggcggcgctgggggaatattacgagcagagagcgacggccggtggatttctgatcactgaaggcactatgatttctccgacttcagctgggtttcctcatgtgccagggattttcacaaaggagcaagtagaggaatggaagaaaatagttgatgtagtgcatgcaaagggtgctgtcatattttgtcagttgtggcatgttggtcgtgcatctcatgaagtgtatcaacctgctggagctgcaccaatatcatctactgagaagcctatatcaaagaggtggagaattctgatgcctgatggaactcatgggatttatccaaaaccaagagcaattggaacctatgagatctcacaagtggttgaagattattgcaggtcggccttgaatgctattgaagcaggttttgatggtattgaaatccatggagctcacggttacttgattgaccaattcttgaaagatgggatcaatgaccggacagatgagtatggtggatcactagccaaccggtgcaaattcatcacacaggtggttcaagcagtcatctcagcaataggagctgatcgtgtaggcgttagagtttcaccagcaatagatcatcttgatgccatggactctaatccactcagcctaggcttagcagttgttgaaagactaaacaaaatccaactccattctggttccaagcttgcctatcttcatgtaacacagccacgatacgtagcatatgggcaaaccgaagcaggcagacttggcagtgaagaggaggaggcgcatttaatgaggactttgaggaacgcatatcaggggacattcatttgcagtggtggatacactagggagctaggaattgaggctgtggcacaaggtgatgctgatctcgtgtcatatggacgtcttttcatctctaatcctgatttggttatgagaatcaagctaaatgcacctctaaataagtataacaggaagacattctatactcaagatccagttgtgggatacacagattaccctttccttcaaggaaatggaagcaacggaccgttatcgcgtctgtga

SEQ ID NO: 22 - protein, 400 aa, Solanum tuberosum
MAKTTSSSAQDGSNPLFSPYKMAKFNLSHRIVLAPMTRCRALNNIPSAALGEYYEQRATAGGFLITEGTMISPTSAGFPHVPGIFTKEQVEEWKKIVDVVHAKGAVIFCQLWHVGRASHEVYQPAGAAPISSTEKPISKRWRILMPDGTHGIYPKPRAIGTYEISQVVEDYCRSALNAIEAGFDGIEIHGAHGYLIDQFLKDGINDRTDEYGGSLANRCKFITQVVQAVISAIGADRVGVRVSPAIDHLDAMDSNPLSLGLAVVERLNKIQLHSGSKLAYLHVTQPRYVAYGQTEAGRLGSEEEEAHLMRTLRNAYQGTFICSGGYTRELGIEAVAQGDADLVSYGRLFISNPDLVMRIKLNAPLNKYNRKTFYTQDPVVGYTDYPFLQGNGSNGPLSRL

SEQ ID NO: 23 - DNA, 1191 bp, Prunus persica
atggcggaggcttcatctcagggacccactctcttttctccgttcaagatgggcaagttcaatctgtctcacagggtggtgcttgcgccgatgacgaggtgccgagcgttgaacggcttgccgcagccggcgctggccgagtactacactcaaaggtcaaccaacggcggctttctgatcaccgaaggcactttggtctccgacactggcgccgggtttccacatgttcctgggatttacaatgatgaacaggtggaggcatggaagaaggtggtggatgccgttcacgccaaaggtgccattattttctgtcaactttggcatgtaggtcgtgcttctcatgaagtttaccaacctggtgggggttcaccaatatcttcaaccgatgttcccatttcgaggaggtggagaattctattaccggatgcgtctcatgccacttaccctaagcctagacgcttagaaacccctgaaatcctccaagtggtggagcattatcgacaggctgccttgaatgccattagagcaggttttgatggaattgagattcatggggcacatggctacctcattgatcaattcttgaaagatgggatcaatgatcgaacagatgagtatggcggatcacttgcaaaccgttgcaaattcttgcttcaggtggttcaagcagtagttggagccgtaggtgctgatagggttggtgtcagaatctcaccagccattgatcaccttgatgcagttgactctgctccacttaccctaagccttggagtgattgaaaggctcaacaagcttcaacaagactggggctcaaaactcacttatctccatgttactcagccccgttacgcagcatatggccaaaccgaatctggcaaacctggcagtgatgaagaggaagctgtgtttatgaggactttaagaaatgcttatcgtggtacatttgttgctagtggtgggtacactagggagcttggaattcatgctgtggcttctagggatgctgatttagtgtcttatggtcgcctttttatctcgaaccccgacttggttttgagattgaagcttaatgcacctttgaccaggtataacaggaagactttctacacgcaagaccctgttgttgggtacacagactacccttttctgagcaatgcaaatgggaaagaggaaccactctcccgcctctga

SEQ ID NO: 24 - protein, 396 aa, Prunus persica
MAEASSQGPTLFSPFKMGKFNLSHRVVLAPMTRCRALNGLPQPALAEYYTQRSTNGGFLITEGTLVSDTGAGFPHVPGIYNDEQVEAWKKVVDAVHAKGAIIFCQLWHVGRASHEVYQPGGGSPISSTDVPISRRWRILLPDASHATYPKPRRLETPEILQVVEHYRQAALNAIRAGFDGIEIHGAHGYLIDQFLKDGINDRTDEYGGSLANRCKFLLQVVQAVVGAVGADRVGVRISPAIDHLDAVDSAPLTLSLGVIERLNKLQQDWGSKLTYLHVTQPRYAAYGQTESGKPGSDEEEAVFMRTLRNAYRGTFVASGGYTRELGIHAVASRDADLVSYGRLFISNPDLVLRLKLNAPLTRYNRKTFYTQDPVVGYTDYPFLSNANGKEEPLSRL

SEQ ID NO: 25 - DNA, 1209 bp, Gossypium hirsutum
atggagcatggagaaaaagtaaaaatggcggattctcaagaaacccctacgctgttctctccttacaagatgggcaaattcaatctttcccacagggtggtgctagcgcctatgacgagatgcagggcgttgaatggaattccaaggccggcgcttgctgaatattacacgcagaggtccactcctggcggctttctcatcactgaaggaacgttgatctccgacactggagcagggtttccacatgttcctggaatctacaatgaagaacaggtggaggcatggaagatgattgtggatgctgttcatgccaaagggggcatcattttctgtcaactatggcatgttggccgagcatctcatacagtgtatcaacctggcggagtggcaccaatatcctcaacaaacaagcccatctcaaagaggtggagaattcttatgccagatggtagctatggcatatatcccaaacctcgacccctggaaacatcagaaatacaagaggttgtagagcattaccgcaaagcagccttgaatgccattcgagcaggttttgatgggattgagattcatggagcacatggttatctcatcgaccaattcttaaaagatgggatcaatgatcgcacagatgagtacggtggatcattggcaaaccgctgcaaattcttaatgcaaattgttcaagcagtagcttcagccattggtatagatagagttgcggtcagaatgtcgcctgcaattgatcacctcgatgcaaccgactctaatccgctcaacctaggcttggctgtgattgagagacttaacaagctccagctacagctggggtcaaaactcgcttatcttcatgtgacgcaacctcgttatcatgcatacgggcaaactgaatcaggcaaacacgggaatgaagacgaggaagcttatttattgagggcactgaagcggacctatcacggaactttcatgtgtagtggcgggttcaatagggagctgggaatgcaagctgtggccgagggtgatgcagatcttgtatcttatggccgccttttcatctcaaatcctgacctagtctttaggttgaaggtcaatgcaccattaaataggtacattaggaagacgttctatactcatgatcctgttgttgggtacacagactatccattcctgaacgaagagaagggtagacaagtactgtcacgcctttga

SEQ ID NO: 26 - protein, 402 aa, Gossypium hirsutum
MEHGEKVKMADSQETPTLFSPYKMGKFNLSHRVVLAPMTRCRALNGIPRPALAEYYTQRSTPGGFLITEGTLISDTGAGFPHVPGIYNEEQVEAWKMIVDAVHAKGGIIFCQLWHVGRASHTVYQPGGVAPISSTNKPISKRWRILMPDGSYGIYPKPRPLETSEIQEVVEHYRKAALNAIRAGFDGIEIHGAHGYLIDQFLKDGINDRTDEYGGSLANRCKFLMQIVQAVASAIGIDRVAVRMSPAIDHLDATDSNPLNLGLAVIERLNKLQLQLGSKLAYLHVTQPRYHAYGQTESGKHGNEDEEAYLLRALKRTYHGTFMCSGGFNRELGMQAVAEGDADLVSYGRLFISNPDLVFRLKVNAPLNRYIRKTFYTHDPVVGYTDYPFLNEEKGRQVLSRL

SEQ ID NO: 27 - DNA, 1200 bp, Coffea canephora
atggctgaaactaagtcagatcaaggaagcccatctctcttttctccatacaagatgggaaagttcaatctgtctcacagggtggttctggcgccgatgacaagatgcagggccataaatagcattcctcagcctgccatggcggagtactacgcccaaagagcaaccaatggtggctttctcatcacggagggcaccatgatctccccaagtgctgccgggtttccgcatgtgccggggatctttacaaaggaacaagtggaggcatggaagcaagtggttgatgcagtacatgccaagggtgctattattttctgtcaactgtggcacgttggccgtgcatcacatgaagtttatcaacctggtggtggtgcacccatatcatcaacgggaaagcctatatcaaagaggtggaggatattgatgcctgatggcagccatgggatctaccctaaaccacgtccattaacaacagcgcatgagattgcgcaagttgtggaagattaccgccagtcggccttgaatgccattgaagccggttttgatggtattgaaatccatggagcacatggctacctaattgaccagttcttgaaagatgggatcaatgatcggacagatgaatatggtggatctgttgcaaatcgctgcaaattcattgtgcaggtggttcaggctgttgtttcagcaattggtgcagatcgtgttggtgtcagaatttcccctgctattgaccatcttgatgccatggactctgatccactaagcttaggcctggcagtgattgagagacttaacgagctccaactgaattcaggctccaagttaacatacttgcacgtgactcaacctcgatatacagcgtatggccagacagaggcaggcagacaggggagtgaagaagaggaggcccaactagtgaggaccttgcgaaaagcttatcaaggaactttcatttccagtggtgggttcaccagagagctaggagttgaagcggtagctcagggtgatgctgatttggtttcctatggtcgcctttttatctcaaatccagacttagttttacgctttaagctaaatgctcctttgattaggtataatagatctaccttctatactcatgatcctgttgtaggatacacagattacccttttctaagcaatggtaccagtggcaatgtaccacaatcacgtctgtaa

SEQ ID NO: 28 - protein, 399 aa, Coffea canephora
MAETKSDQGSPSLFSPYKMGKFNLSHRVVLAPMTRCRAINSIPQPAMAEYYAQRATNGGFLITEGTMISPSAAGFPHVPGIFTKEQVEAWKQVVDAVHAKGAIIFCQLWHVGRASHEVYQPGGGAPISSTGKPISKRWRILMPDGSHGIYPKPRPLTTAHEIAQVVEDYRQSALNAIEAGFDGIEIHGAHGYLIDQFLKDGINDRTDEYGGSVANRCKFIVQVVQAVVSAIGADRVGVRISPAIDHLDAMDSDPLSLGLAVIERLNELQLNSGSKLTYLHVTQPRYTAYGQTEAGRQGSEEEEAQLVRTLRKAYQGTFISSGGFTRELGVEAVAQGDADLVSYGRLFISNPDLVLRFKLNAPLIRYNRSTFYTHDPVVGYTDYPFLSNGTSGNVPQSRL

SEQ ID NO: 29 - DNA, 1414 bp, Glycine max
  CDS (1)..(1167)
  3′UTR (1168)..(1414)
atggcagataactcaattagcttattttctccatacaacaagatgggcaaattcaacctctctcatagggtggtattggctcccatgaccagatgcagagcgctcaatgggactccactggcagcacatgctgaatactacgctcagagatcaacaccgggtggatttctcatcactgaaggcaccttgatctctccaacttcttctgggtttcctcatgttcctggaatatactcagatgaacaggtagaggcatggagaaatgtagtggacgccgtgcatgccaacggcagctttatcttctgtcaactctggcatgttggccgtgcatcacatccagtgtatcagcctggtggggctctaccctcttcgtccaccagcaaacccatatcagacaagtggaaaattctcatgcccgatggctcccatggcatctatccagagcctcgtgcacttaccacttctgagatatctgaaatagtgcatcattatcgccaagcagctattaatgcaattcgagcaggttttgatggaatcgagattcatggagcacatgggtatctcattgatcaattcttaaaggatgcaatcaatgatagaacagatgaatacggtggaccactagaaaaccggtgcaggttcttaatggaggtagttgaagctgttgtctctgccattggagcggaaagagttgctatcagaatttcaccagcaattgatttcaatgacgcctttgactctgacccacttgggctaggcttagcagtgattgaaagactcaacaatttgcagaaacaagtgggcacaaaactcgcttatcttcatgttactcagcctcgattcacacttttggcgcaaaccgagtcagtgagtgaaaaggaggaagctcatttcatgcagaaatggagagaggcttatgagggaacattcatgtgtagtggagcttttactagggactcaggaatggaagctgtagctgaaggccatgctgatttggtatcctatggtcgtcttttcatctccaatccagacttggttttaaggcttaagctcaatgcacctcttaccaagtataacaggaacacattttacacccaagatcctgttataggctacacagattatcctttctttaatggaacaactgagacaaaattaagtaactagctaaggccatgcatgccctttaattttaatctccatatggctttttgaataataatgttcataacattcaaaactcttcagttgagtttatcctcagacaaacaaattaagtggttcattcacttgttagggtatttagatcttaggttaattagtctccggcattttgatttcatttcaatttgtattcagtctttcattttgaataaaataatattaagttttttgccttaaaaaaaaaaaaaaa

SEQ ID NO: 30 - protein, 388 aa, Glycine max
MADNSISLFSPYNKMGKFNLSHRVVLAPMTRCRALNGTPLAAHAEYYAQRSTPGGFLITEGTLISPTSSGFPHVPGIYSDEQVEAWRNVVDAVHANGSFIFCQLWHVGRASHPVYQPGGALPSSSTSKPISDKWKILMPDGSHGIYPEPRALTTSEISEIVHHYRQAAINAIRAGFDGIEIHGAHGYLIDQFLKDAINDRTDEYGGPLENRCRFLMEVVEAVVSAIGAERVAIRISPAIDFNDAFDSDPLGLGLAVIERLNNLQKQVGTKLAYLHVTQPRFTLLAQTESVSEKEEAHFMQKWREAYEGTFMCSGAFTRDSGMEAVAEGHADLVSYGRLFISNPDLVLRLKLNAPLTKYNRNTFYTQDPVIGYTDYPFFNGTTETKLSN